(19) United States
(12) Patent Application Publication
(10) Pub. No.: US 2010/0226981 A1
(43) Pub. Date: Sep. 9, 2010

(54) ORAL DOSAGE FORMS HAVING A HIGH LOADING OF A GABAPENTIN PRODRUG

(75) Inventors: Sami Karaborni, Cupertino, CA (US); Laura E. Maurer, Sunnyvale, CA (US); David J. Kidney, San Jose, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: XenoPort, Inc., Santa Clara, CA (US)

(21) Appl. No.: 12/718,857

(22) Filed: Mar. 5, 2010

Related U.S. Application Data
(60) Provisional application No. 61/158,065, filed on Mar. 6, 2009.

(51) Int. Cl. A61K 9/36; A61K 31/195; A61P 25/00; A61P 29/00; A61P 11/00; A61P 13/10

(52) U.S. Cl. 424/465; 514/561

(57) ABSTRACT

Sustained release oral dosage forms with a high loading of a gabapentin prodrug are disclosed.

This application claims the benefit of U.S. Provisional Patent Application No. 61/158,065 filed Mar. 6, 2009, which is incorporated by reference herein for all purposes.

### FIELD

Methods provided by the present disclosure relate to sustained release oral dosage forms with a high loading of a gabapentin prodrug.

### BACKGROUND

1-([(α-Isobutanoyloxyethoxy)carbonyl]aminomethyl)-1-cyclohexane acetic acid (1),

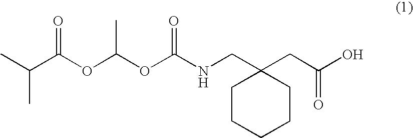

a prodrug of the GABA analog gabapentin, 1-(aminomethyl)cyclohexaneacetic acid, exhibits high bioavailability as gabapentin when dosed either orally or directly into the colon of a mammal (Cundy et al., J Pharm Expt'l Ther 2004, 311(1), 315-323; Cundy et al., J Pharm Expt'l Ther 2004, 311(1), 324-333; Cundy et al., 60^{th} American Academy Neurology Annual Meeting, Chicago, Ill., Apr. 12-19, 2008, Poster PO 5.168; and Cundy et al., J Clin Pharmacol 2008, 48(12), 1378-88). The high gabapentin oral bioavailability following administration of compound (1) favors the use of compound (1) in oral dosage forms, including sustained-release oral dosage forms, and the use of such oral dosage forms for treating epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, and vulvodynia.

The synthesis of compound (1) is described by Gallop et al., U.S. Pat. Nos. 6,818,787, 7,186,855, 7,227,028, and 6,927,036; Estrada et al., US 2005-0154057; Bhat et al., US 2005-0070715; Raillard et al., U.S. Pat. No. 7,332,924, and U.S. application Ser. Nos. 12/537,764 and 12/537,798. A crystalline form of compound (1) is described by Estrada et al., US 2005-0154057.

Oral dosage forms comprising compound (1) are disclosed in Cundy and Gallop, U.S. Pat. No. 6,833,140; and Cundy et al., US 2006-0141034. Current tablet formulations have a compound (1) loading that results in large tablets to support a high drug dose, and the properties of the granulation used to prepare the tablets are not ideally suited for commercial tableting operations.

### SUMMARY

Oral tablet dosage forms having a high loading of 1-([α-isobutanoyloxyethoxy)carbonyl]aminomethyl)-1-cyclohexane acetic acid or pharmaceutically acceptable salt thereof prepared from granulations with greater than 95 wt-% 1-([(α-isobutanoyloxyethoxy)carbonyl]aminomethyl)-1-cyclohexane acetic acid are disclosed.

In a first aspect, oral tablet dosage forms are disclosed comprising about 80 wt-% to about 95 wt-% 1-([(α-isobutanoyloxyethoxy)carbonyl]aminomethyl)-1-cyclohexane acetic acid or pharmaceutically acceptable salt thereof.

In a second aspect, solid granulations comprising greater than about 95 wt-% 1-([(α-isobutanoyloxyethoxy)carbonyl]aminomethyl)-1-cyclohexane acetic acid or pharmaceutically acceptable salt thereof are disclosed.

In a third aspect, oral tablet dosage forms comprising solid granulations comprising greater than about 95 wt-% 1-([α-isobutanoyloxyethoxy)carbonyl]aminomethyl)-1-cyclohexane acetic acid or pharmaceutically acceptable salt thereof are disclosed.

In a fourth aspect, methods of treating a disease in a patient are disclosed wherein the disease is chosen from epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, and vulvodynia, comprising orally administering to a patient in need of such treatment at least one oral tablet dosage form provided by the present disclosure.

### BRIEF DESCRIPTION OF THE DRAWINGS

Those skilled in the art will understand that the drawings, described herein, are for illustration purposes only. The drawings are not intended to limit the scope of the present disclosure.

FIG. 1 shows dissolution profiles for 1200 mg compound (1) tablets having different weight percent METHOCEL™-K100M.

FIG. 2 shows dissolution profiles for 600 mg tablets having different weight percent METHOCELT™-K100M.

FIG. 3 shows dissolution profiles for 600 mg tablets having different weight percent METHOCELT™-K4M.

FIG. 4 shows dissolution profiles for different sized 1200 mg tablets having 7 wt-% METHOCEL™-K100M and 2 wt-% magnesium stearate.

FIG. 5 shows dissolution profiles for 600 mg tablets of different hardness having 8 wt-% METHOCEL™-100M and 2 wt-% magnesium stearate.

FIG. 6 shows dissolution profiles for 600 mg tablets of different hardness having 8 wt-% METHOCELT™-K100M and 3 wt-% magnesium stearate.

FIG. 7 shows the strength of granules having 97 wt-% compound (1) and 3 wt-% METHOCELT™-E4M prepared using different amounts of water during granulation.

FIG. 8 shows the strength of granules having 98 wt-% compound (1), 1 wt-% METHOCELT™-E4M, and 1 wt-% sodium lauryl sulfate prepared using different amounts of water during granulation.

FIG. 9 shows the force of ejection from the tooling die for 600 mg tablets having different weight percent magnesium stearate.

FIG. 10 shows dissolution profiles for 600 mg tablets of different hardness and different weight percent magnesium stearate.

FIG. 11 shows the mean pharmacokinetic profile for gabapentin in the plasma of ten (10) healthy, adult male, fasted human subjects following administration of 1200 mg tablet prepared as described in Example 5.

### DETAILED DESCRIPTION

### Definitions

“AUC” is the area under a curve representing the concentration of a compound or metabolite thereof in the blood or plasma of a patient as a function of time following administration of the compound to the patient. For example, the administered compound can be the gabapentin prodrug (1) and the corresponding metabolite gabapentin. The AUC may be determined by measuring the concentration of a compound or metabolite thereof in blood using methods such as liquid chromatography-tandem mass spectrometry (LC/MS/MS), at various time intervals, and calculating the area under the blood or plasma concentration-versus-time curve. The concentration versus time curve is also referred to as the pharmacokinetic profile. Suitable methods for calculating the AUC from a drug concentration-versus-time curve are well known in the art. For example, an AUC for gabapentin may be determined by measuring the concentration of gabapentin in the blood of a patient following administration of a gabapentin prodrug, such as compound (1), to the patient. AUC₀₋₂₄ is the area under the curve from administration (time 0) to 24 hours following administration. AUC_{ss,24} is the area under the curve over a 24 hour period following a dosing regimen administered over a period of days (steady state). AUC_{inf} is the AUC value extrapolated to infinity (AUC_{inf}) calculated as AUC_{inf}=AUC₍₀₋ₜₗₐₛₜ₎+Cₗₐₛₜ/λ_{z}, where tₗₐₛₜ is the time of the last quantifiable concentration (Cₗₐₛₜ) and λ_{z} is the rate constant of the apparent terminal elimination phase.

“Bioavailability” refers to the rate and amount of gabapentin that reaches the systemic circulation of a patient following administration of the compound (1) to the patient and can be determined by evaluating, for example, the blood or plasma concentration-versus-time profile for gabapentin. Parameters useful in characterizing a blood concentration-versus-time curve include the area under the curve (AUC), the time to peak concentration (Tₘₐₓ), and the maximum gabapentin concentration (Cₘₐₓ), where Cₘₐₓ is the maximum concentration of a drug in the blood plasma of a patient following administration of a dose of compound (1) to the patient, and T_{in a}, is the time to the maximum concentration (Cₘₐₓ) of gabapentin in the blood or plasma of a patient following administration of a dose of compound (1) to the patient.

Absolute oral bioavailability is the bioavailability of a compound or metabolite thereof following oral administration compared to the bioavailability following intravenous administration of an equivalent amount of the compound or metabolite thereof. Relative oral bioavailability of a compound or metabolite thereof is the bioavailability following oral administration of a compound or metabolite thereof relative to administration of an equivalent amount of the compound or metabolite thereof in another dosage form and/or route of administration. For example, in certain embodiments, relative oral bioavailability expressed as % Fᵣₑ₁ is the bioavailability of gabapentin determined by the AUC₀₋₂₄ following oral administration of compound (1) to a patient relative to the bioavailability of gabapentin following oral administration of 20 mg compound (1) as a sustained release dosage form.

“Bioequivalence” refers to equivalence of the rate and extent of absorption of gabapentin after administration of equal doses of gabapentin or compound (1) to a patient. As used herein, two pharmacokinetic profiles are bioequivalent if the 90% confidence interval for the ratio of the mean response of the two profiles is within the limits of 0.8 and 1.25. The mean response includes at least one of the characteristic parameters of a profile such as Cₘₐₓ, Tₘₐₓ, and AUC.

“Compound (1)” includes the gabapentin prodrug (1), 1-([α-isobutanoyloxyethoxy)carbonyl]aminomethyl)-1-cyclohexane acetic acid (IUPAC name [1-({[({1-[(2-methylpropanoyl)oxy]ethyl}oxy)carbonyl]amino}methyl)cyclohexyl]acetic acid) and pharmaceutically acceptable salts thereof. Compound (1) may refer to the racemate (±)-1-([(α-isobutanoyloxyethoxy)carbonyl]aminomethyl)-1-cyclohexane acetic acid, and/or either of the two enantiomers. “Compound (1)” is used interchangeably with “gabapentin prodrug (1)”. In certain embodiments, gabapentin prodrug (1)/compound (1) is the free acid. In certain embodiments, gabapentin prodrug (1)/compound (1) is the hydrochloride salt. Compound (1)/gabapentin prodrug (1) is also referred to as gabapentin enacarbil, XP13512 or GSK 183262.

Compound (1) may also exist in several tautomeric forms including the enol form, the keto form, and mixtures thereof. Accordingly, the chemical structure depicted herein encompasses all possible tautomeric forms of the illustrated compounds.

Compound (1) may exist in unsolvated forms as well as solvated forms, including hydrated forms and as N-oxides. In general, compound (1) may be free acid, hydrated, solvated, N-oxides, or combinations of any of the foregoing. Compound (1) may exist in crystalline, co-crystalline, or amorphous forms. Compound (1) includes pharmaceutically acceptable salts thereof, or pharmaceutically acceptable solvates of the free acid form of any of the foregoing, as well as crystalline forms of any of the foregoing.

Certain pharmaceutically acceptable salts of compound (1) may exist in the form of solvates. A solvate refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Compounds of the present disclosure” include any compounds falling within the scope of Formula (1)/compound (1)/gabapentin prodrug (1). Compounds may be identified either by their chemical structure and/or chemical name. In the event the chemical structure and chemical name conflict, the chemical structure is determinative of the identity of the compound. The compounds described herein comprise a chiral center. Unless specifically indicated, any chemical structures depicted with a relative configuration encompass all possible enantiomers of the illustrated compounds including the enantiomerically pure form and enantiomeric mixtures. Enantiomeric may be resolved into their component enantiomers o using separation techniques or chiral synthesis techniques well known to the skilled artisan. For example, resolution of the enantiomers may be accomplished, for example, by conventional methods such as crystallization in the presence of a resolving agent, or chromatography, using, for example a chiral high-pressure liquid chromatography (HPLC) column.

Compound (1) may also include isotopically labeled compounds where one or more atoms have an atomic mass different from the atomic mass conventionally found in nature. Examples of isotopes that may be incorporated into the compound include, but are not limited to, ²H, ³H, ¹¹C, ¹³C, ¹⁴C, ¹⁵N, ¹⁸O, ¹⁷O, etc. In certain embodiments, compound (1) is not isotopically labeled.

Compound (1) may exist in crystalline, co-crystalline, or amorphous forms.

“Cₘₐₓ” is the maximum gabapentin concentration observed in the blood of a patient following administration of a dose of compound (1) to the patient.

“C₁₂” is the gabapentin concentration observed in the blood of a patient twelve (12) hours after administration of compound (1) to the patient.

“Tₘₐₓ” is the time to the maximum concentration (Cₘₐₓ) of gabapentin in the blood of a patient following administration of a dose of compound (1) to the patient.

“T_{1/2}” is the time interval between Tₘₐₓ and the time at which the gabapentin concentration in the blood of a patient has decreased to one-half the maximum drug concentration.

“Dosage form” refers to a form of a formulation that contains an amount of active agent or prodrug of an active agent, i.e., gabapentin prodrug (1), which can be administered to a patient to achieve a therapeutic effect. An oral dosage form is intended to be administered to a patient via the mouth and swallowed. A dose of a drug may include one or more dosage forms administered simultaneously or over a period of time.

“Patient” includes mammals, such as for example, humans.

“Pharmaceutically acceptable” refers to approved or approvable by a regulatory agency of a federal or a state government, listed in a U.S. Pharmacopeia, or listed in other generally recognized pharmacopeia for use in mammals, including humans.

“Dose of 1-([(α-isobutanoyloxyethoxy)carbonyl]aminomethyl)-1-cyclohexane acetic acid” refers to the amount, typically in milligrams, of 1-([α-isobutanoyloxyethoxy)carbonyl]aminomethyl)-1-cyclohexane acetic acid not including the weight of the salt or solvent if the 1-([α-isobutanoyloxyethoxy)carbonyl]aminomethyl)-1-cyclohexane acetic acid is a salt or solvate.

“Pharmaceutically acceptable salt” refers to a salt of a compound such as compound (1) that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound either is replaced by a metal ion, e.g., an alkali metal ion, an alkaline earth metal ion, or an aluminum ion; or coordinates with an organic base such as ethanolamine, diethanolamine, triethanolamine, N-methylglucamine, and the like. In certain embodiments, a salt of compound (1) is the hydrochloride salt, and in certain embodiments, the sodium salt.

“Pharmaceutically acceptable vehicle” or “pharmaceutically acceptable excipient” refers to a pharmaceutically acceptable diluent, a pharmaceutically acceptable adjuvant, a pharmaceutically acceptable excipient, a pharmaceutically acceptable carrier, or a combination of any of the foregoing with which a compound such as the gabapentin prodrug, 1-([(α-isobutanoyloxyethoxy)carbonyl]aminomethyl)-1-cyclohexane acetic acid (1), may be administered to a patient, which does not destroy the pharmacological activity thereof, and which is nontoxic when administered in doses sufficient to provide a therapeutically effective amount of the gabapentin prodrug or gabapentin metabolite.

“Pharmaceutical composition” refers to a composition comprising gabapentin prodrug (1) and at least one pharmaceutically acceptable vehicle, with which the prodrug is to be administered to a patient.

“Prodrug” refers to a derivative of an active compound (drug) that undergoes a transformation under the conditions of use, such as within the body, to release an active drug. Prodrugs are frequently, but not necessarily, pharmacologically inactive until converted into the active drug. Prodrugs can be obtained by bonding a promoiety (defined herein), typically via a functional group, to a drug. For example, gabapentin prodrug (1) is metabolized within a patient's body to form the parent drug gabapentin.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach or the agent may be supplied exogenously. For example, for gabapentin prodrug (1), the drug is gabapentin and the promoiety has the structure:

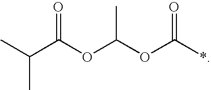

“Sustained release” refers to release of a compound from a dosage form at a rate effective to achieve a therapeutic amount of the compound, or active metabolite thereof, in the systemic blood circulation over a prolonged period of time relative to that achieved by oral administration of an immediate formulation of the compound.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The therapeutically effective amount may vary depending, for example, on the compound, the disease, disorder, and/or symptoms of the disease, severity of the disease or disorder, and/or symptoms of the disease or disorder, the age, weight, and/or health of the patient to be treated, and the judgment of the prescribing physician. A therapeutically effective amount may be ascertained by those skilled in the art or capable of determination by routine experimentation.

“Treating” or “treatment” of any disease refers to arresting or ameliorating a disease or at least one of the clinical symptoms of a disease, reducing the risk of acquiring a disease or at least one of the clinical symptoms of a disease, arresting or retarding the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease even though that patient does not yet experience or display symptoms of the disease. Prophylaxis refers to prevention of a disease.

Reference is now made in detail to certain embodiments of dosage forms and methods. The disclosed embodiments are not intended to be limiting of the claims. To the contrary, the claims are intended to cover all alternatives, modifications, and equivalents.

### Composition

Sustained release oral dosage forms provided by the present disclosure comprise compound (1) and pharmaceutically acceptable excipients. 1-([(α-Isobutanoyloxyethoxy)carbonyl]aminomethyl)-1-cyclohexane acetic acid as given by the following structure:

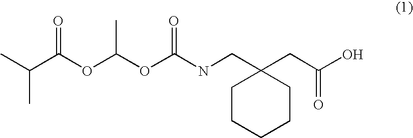

exhibits high oral bioavailability of gabapentin at doses greater than about 400 mg-equivalents gabapentin. Compound (1) includes 1-([α-isobutanoyloxyethoxy)carbonyl]aminomethyl)-1-cyclohexane acetic acid and pharmaceutically acceptable salts thereof. In certain embodiments, compound (1) is the free acid form of 1-([(α-isobutanoyloxyethoxy)carbonyl]aminomethyl)-1-cyclohexane acetic acid. In certain embodiments, compound (1) is crystalline, and in certain embodiments, is the crystalline form of the free acid of 1-([(α-Isobutanoyloxyethoxy)carbonyl]aminomethyl)-1-cyclohexane acetic acid. In certain embodiments, compound (1) in the dosage form is the crystalline form disclosed in Estrada et al., US 2005-0154057. In certain embodiments, crystalline 1-{[α-isobutanoyloxyethoxy)carbonyl]-aminomethyl}-1-cyclohexane acetic acid exhibits characteristic scattering angles at 7.0°±0.3°, 8.2°±0.3°, 10.5°±0.3°, 12.8°±0.3°, 14.9°±0.3°, and 16.4°±0.3° in an X-ray powder diffractogram using Cu Kαradiation. In certain embodiments, crystalline 1-{[(α-isobutanoyloxyethoxy)carbonyl]-aminomethyl}-1-cyclohexane acetic acid exhibits characteristic scattering angles at 7.0°±0.3°, 8.2°±0.3°, 10.5°±0.3°, 12.8°±0.3°, 14.9°±0.3°, 16.4°±0.3°, 17.9°±0.3°, 18.9°±0.3°, 20.9°±0.3°, 23.3°±0.3°, 25.3°±0.3°, and 26.6°±0.3° in the X-ray powder diffractogram using Cu Kα radiation. In certain embodiments, crystalline 1-{[α-isobutanoyloxyethoxy)carbonyl]-aminomethyl}-1-cyclohexane acetic acid exhibits characteristic peaks at 7.0°±0.3°, 8.2°±0.3°, 10.5°±0.3°, 12.8°±0.3°, 14.9°±0.3°, 16.4°±0.3°, 18.1°±0.3°, 18.9°±0.3°, 20.9°±0.3°, 23.3°±0.3°, 25.3°±0.3°, and 26.6°±0.3° in the X-ray powder diffractogram using Cu Kα radiation. In certain embodiments, crystalline 1-{[(α-isobutanoyloxyethoxy)carbonyl]-aminomethyl}-1-cyclohexane acetic acid exhibits characteristic peaks at 7.0°±0.3°, 8.2°±0.3°, 10.5°±0.3°, 12.8°±0.3°, 14.9°±0.3°, 16.4°±0.3°, 17.9°±0.3°, 18.1°±0.3°, 18.9°±0.3°, 20.9°±0.3°, 23.3°±0.3°, 25.3°±0.3°, and 26.6°±0.3° in the X-ray powder diffractogram using Cu Kα radiation. In certain of any of the preceding embodiments, crystalline 1-{[(α-isobutanoyloxyethoxy)carbonyl]-aminomethyl}-1-cyclohexane acetic acid has a melting point range of between about 63° C. and about 64° C. as determined by differential scanning calorimetry at a scan rate of 5° C./minute. In certain of any of the preceding embodiments, crystalline 1-{[(α-isobutanoyloxyethoxy)carbonyl]-aminomethyl}-1-cyclohexane acetic acid has a melting point range of between about 64° C. and about 66° C. as determined by open capillary melting point determination.

Compound (1) may be prepared using the methods described by Gallop et al., U.S. Pat. Nos. 6,818,787, 7,186,855, 7,227,028, and 6,927,036; Estrada et al., US 2005-0154057; Bhat et al., US 2005-0070715; and/or Raillard et al., U.S. Pat. No. 7,332,924, and U.S. application Ser. Nos. 12/537,764 and 12/537,798.

Sustained release oral dosage forms provided by the present disclosure comprise compound (1) and one or more pharmaceutically acceptable excipients. Sustained release oral dosage forms may comprise greater than about 80 wt-% compound (1), greater than about 85 wt-% compound (1), greater than about 90 wt-% compound (1), or in certain embodiments, greater than about 95 wt-% compound (1), where wt-% is based on the total weight of the dosage form. In certain embodiments, an oral dosage from comprises from about 85 wt-% to about 95 wt-% compound (1). In certain embodiments, a dosage from may contain from about 300 mg to about 1300 mg compound (1), for example, about 600 mg compound (1) or about 1200 mg compound (1).

In certain embodiments, the one or more pharmaceutically acceptable excipients is hydroxypropylmethyl cellulose (HPMC) and a pharmaceutically acceptable lubricant. The amount of HPMC in a dosage form may be from about 3 wt-% to about 18 wt-%, from about 6 wt-% to about 9 wt-% and in certain embodiments, from about 7 wt-% to about 8 wt-% hydroxypropylmethyl cellulose. In certain embodiments, the hydroxypropylmethyl cellulose is chosen from a hypromellose 2208 polymer characterized by a methoxyl content of 19% to 24% and a hydroxypropyl content of 7% to 12% such as, for example, METHOCEL™ K3, METHOCEL™ K100, METHOCEL™ K4M, METHOCEL™ K15M, and METHOCEL™ K100M (Dow Chemical), or other chemically equivalent polymer. In certain embodiments, the hydroxypropylmethyl cellulose is a hypromellose 2208 polymer having a viscosity of 80,000 cps to 120,000 cps in a 2% aqueous solution. In certain embodiments, the hydroxypropylmethyl cellulose is chosen from a hypromellose 2208 polymer having a methoxyl content of 19% to 24%, a hydroxypropyl content of 7% to 12%, and a viscosity of 80,000 cps to 120,000 cps in a 2% aqueous solution, such as METHOCEL™ K100M.

The amount of lubricant in a dosage form may be from about 0.5 wt-% to about 4 wt-%, from about 2 wt-% to about 4 wt-%, and in certain embodiments is about 3 wt-%. In certain embodiments, the lubricant may be chosen from magnesium stearate, sodium stearyl fumarate, and stearic acid; and in certain embodiments, the lubricant is magnesium stearate.

Sustained release oral dosage forms provided by the present disclosure have a high loading of 1-{[(α-isobutanoyloxyethoxy)carbonyl]-aminomethyl}-1-cyclohexane acetic acid, e.g., greater than about 80 wt-% compound (1). Compound (1) is provided in the form of granules having a high loading of compound (1). For example, the granules may comprise greater than about 95 wt-% compound (1), greater than about 96 wt-% compound (1), greater than about 97 wt-% compound (1), greater than about 98 wt-% compound (1), and in certain embodiments, greater than about 99 wt-% compound (1). The granules may further comprise a release rate-controlling polymer such as a hydroxypropylmethyl cellulose and a surfactant.

Granules may comprise from about 0.5 wt-% to about 1.5 wt-%, and in certain embodiments about 1 wt-% of a polymer such as hydroxypropylmethyl cellulose, which may function as a release rate-controlling polymer and/or as a binder. The hydroxypropylmethyl cellulose may be chosen from a hypromellose 2910 characterized by a methoxyl content of 28% to 30% and a hydroxypropyl content of 7% to 12%, such as, for example, METHOCEL™ E3, METHOCEL™ E5, METHOCEL™ E6, METHOCEL™ E15, METHOCEL™ E50, METHOCEL™ E4M, and METHOCEL™ E10 (Dow Chemical), or other chemically equivalent polymer. In certain embodiments, the hydroxypropylmethyl cellulose is a hypromellose 2910 polymer having a viscosity of 3,000 cps to 5,600 cps in a 2% aqueous solution. In certain embodiments, the hydroxypropylmethyl cellulose is chosen from a hypromellose 2910 polymer having a methoxyl content of 28% to 30%, a hydroxypropyl content of 7% to 12%, and a viscosity of 3,000 cps to 5,600 cps in a 2% aqueous solution, such as METHOCEL™ E4M.

Granules may comprise from about 0.5 wt-% to about 1.5 wt-% surfactant or in certain embodiments, about 1 wt-% surfactant. A surfactant may be chosen from sodium lauryl sulfate, poloxamers (triblock copolymers of poly(propylene oxide) and poly(ethylene oxide)), and polysorbates (polyethylene derivatives of sorbitan monolaurate). In certain embodiments, the surfactant is sodium lauryl sulfate.

In certain embodiments, granules consist essentially of about 98 wt-% 1-([(α-isobutanoyloxyethoxy)carbonyl]aminomethyl)-1-cyclohexane acetic acid; about 1 wt-% of a surfactant; and about 1 wt-% of a hypromellose 2910 polymer having a methoxyl content of 28-30%, a hydroxypropyl content of 7-12%, and a viscosity of 3,000 cps to 5,600 cps in a 2% aqueous solution.

Granules having a high loading of compound (1) may be prepared using high shear wet granulation. At least in part, the amount of release rate-controlling polymer/binder and surfactant used to form the granules is chosen to provide a wide processing window for the amount of water used during granulation. It is generally desirable to be able to vary process parameters without significantly negatively impacting the properties of the granules and to produce granules having optimal flow and mechanical properties to facilitate subsequent tableting processes. Thus, granules provided by the present disclosure may be prepared using water equivalent to from about 20 wt-% to about 30 wt-% of the dry formulation used to prepare the granules, from about 23 wt-% to about 29 wt-% of the dry formulation used to prepare the granules, and in certain embodiments, from about 26 wt-% to about 28 wt-% of the dry formulation used to prepare the granules.

Alternatively, granules comprising a high loading of compound (1) may be prepared using roller compaction.

Alternatively, granules comprising a high loading of compound (1) may be prepared using fluid bed granulation.

Granules provided by the present disclosure, such as for example, granules comprising compound (1), METHOCEL™ E4M, and sodium lauryl sulfate, exhibit a Flodex less than about 20 mm, less than about 18 mm, and in certain embodiments, less than about 10 mm.

When the constituents of the granules are included, in certain embodiments, oral dosage forms provided by the present disclosure comprise from about 80 wt-% to about 90 wt-% compound (1), from about 0.8 wt-% to about 1.0 wt-% of a first hydroxypropylmethyl cellulose polymer, from about 0.8 wt-% to about 1.0 wt-% surfactant, from about 3 wt-% to about 15 wt-% of a second hydroxypropylmethyl cellulose polymer, and from about 0.5 wt-% to about 3.5 wt-% of a lubricant. In certain embodiments, oral dosage forms provided by the present disclosure comprise from about 85 wt-% to about 90 wt-% compound (1), from about 0.8 wt-% to about 1.0 wt-% of a hypromellose 2910 polymer having a methoxyl content of 28% to 30%, a hydroxypropyl content of 7% to 12%, and a viscosity of 3,000 cps to 5,600 cps in a 2% aqueous solution, from about 0.8 wt-% to about 1.0 wt-% sodium lauryl sulfate, from about 6 wt-% to about 9 wt-% of hypromellose 2208 polymer having a methoxyl content of 19% to 24%, a hydroxypropyl content of 7% to 12%, and a viscosity of 80,000 cps to 120,000 cps in a 2% aqueous solution, and from about 2.5 wt-% to about 3.5 wt-% of magnesium stearate. In certain embodiments, oral dosage forms provided by the present disclosure comprise from about 85 wt-% to about 90 wt-% compound (1), from about 0.8 wt-% to about 1.0 wt-% of METHOCEL™-E4M, from about 0.8 wt-% to about 1.0 wt-% sodium lauryl sulfate, from about 6 wt-% to about 9 wt-% of METHOCEL™-K100M, and from about 2.5 wt-% to about 3.5 wt-% of magnesium stearate.

### Dosage Forms

Sustained release oral dosage forms provided by the present disclosure may be provided as tablets. Formulations used to prepare the tablets comprise a blend of one or more pharmaceutically acceptable excipients and granules comprising a high loading of compound (1) and one or more pharmaceutically acceptable excipients. In certain embodiments, the granules are prepared by high shear wet granulation methods. Formulations provided by the present disclosure are generally useful in forming oral tablet dosage forms by tablet compression.

In certain embodiments, dosage forms may be in the form of tablets comprising compound (1). Tablet dosage forms may be of any shape suitable for oral administration of a drug such as spheroidal, cube-shaped, oval, or ellipsoidal. In certain embodiments, tablet dosage forms, e.g., an oral dosage form in the form of a tablet, provided by the present disclosure are matrix systems in which the gabapentin prodrug (1) is dispersed in a matrix comprising at least one release-rate modifying compound. Matrix systems are well-known in the art as described, for example, in “Handbook of Pharmaceutical Controlled Release Technology,” ed. Wise, Marcel Dekker, Inc. (2000) and “Treatise on Controlled Drug Delivery, Fundamentals, Optimization, and Applications,” ed. Kydonieus, Marcel Dekker, Inc. (1992).

In certain embodiments, the amount of compound (1) in a dosage form provided by the present disclosure (dose strength) is from about 100 mg to about 2000 mg, in certain embodiments, from about 300 mg to about 1200 mg, and in certain embodiments is 300 mg, 600 mg, 900 mg, or 1200 mg. For dosage forms comprising a pharmaceutically acceptable salt and/or solvate of compound (1), the amount of compound (1) in a dosage form refers to the mass equivalent weight of compound (1) comprising the salt and/or hydrate. In certain embodiments, tablet dosage forms may comprise a therapeutically effective amount of compound (1). A therapeutically effective amount of compound (1) may comprise from about 50 mg-equivalents to about 1,000 mg-equivalents gabapentin, or from about 150 mg-equivalents to about 600 mg-equivalents gabapentin. One (1) mg compound (1) comprises 0.521 mg-equivalents of gabapentin. In certain embodiments, a therapeutically effective amount of compound (1) is less than an amount that causes adverse effects in a patient such as dizziness, somnolence, fatigue, and/or ataxia.

In certain embodiments in which tablet dosage forms comprise less than a therapeutically effective amount of compound (1), multiple tablet dosage forms may be administered to a patient simultaneously or over a period of time to provide a therapeutically effective dose of compound (1).

In addition to compound (1) and the release rate modifying compounds disclosed herein, tablet dosage forms may also comprise one or more pharmaceutically acceptable vehicles such as surfactants, lubricants, plasticizers, binding agents, diluents, anti-adherents, glidants, buffers, dyes, wetting agents, emulsifying agents, pH buffering agents, stabilizing agents, thickening agents, disintegrants, and coloring agents.

Diluents, or fillers, may be added to increase the bulk to make dosage forms a practical size for compression. Examples of diluents useful in tablet dosage forms provided by the present disclosure include dibasic calcium phosphate dibasic calcium phosphate dihydrate, calcium sulfate, dicalcium phosphate, tricalcium phosphate, lactose, cellulose including microcrystalline cellulose, kaolin, mannitol, sodium chloride, dry starch, pregelatinized starch, compressible sugar, and combinations of any of the foregoing. In certain embodiments, a diluent is selected from dibasic calcium phosphate and microcrystalline cellulose. Fillers may be water insoluble, water soluble, or combinations thereof. Examples of useful water insoluble fillers include silicon dioxide, titanium dioxide, talc, alumina, starch, kaolin, polacrilin potassium, powdered cellulose, microcrystalline cellulose, fumed silica, glyceryl monostearate, magnesium stearate, calcium stearate, colloidal silica, micronized silica, magnesium trisilicate, gypsum, and combinations of any of the foregoing. Examples of water-soluble fillers include water soluble sugars and sugar alcohols, such as lactose, glucose, fructose, sucrose, mannose, dextrose, galactose, the corresponding sugar alcohols and other sugar alcohols, such as mannitol, sorbitol, xylitol, and combinations of any of the foregoing.

Glidants may be included in dosage forms provided by the present disclosure to reduce sticking effects during processing, film formation, and/or drying. Examples of useful glidants include talc, magnesium stearate, glycerol monostearate, colloidal silicon dioxide, precipitated silicon dioxide, fumed silicon dioxide, and combinations of any of the foregoing. In certain embodiments, a glidant is colloidal silicon dioxide.

Binding agents may be included in dosage forms to facilitate adhesion of the constituents. Examples of binding agents useful in tablet dosage forms provided by the present disclosure include polyvinyl acetate phthalate, molasses, methylcellulose, hydroxypropyl cellulose (HPC), hydroxypropylmethyl cellulose (HPMC), sodium carboxymethyl cellulose, microcrystalline cellulose, and polyvinyl pyrrolidone. In certain embodiments provided by the present disclosure, a binder is microcrystalline cellulose such as AVICEL® PH200 (FMC Corporation).

Plasticizers may be included in tablet dosage forms provided by the present disclosure. Examples of plasticizers useful in tablet dosage forms provided by the present disclosure include alkyl citrates such as triethyl citrate, acetyl triethyl citrate, tributyl citrate, acetyl triethyl citrate, and acetyl tributyl citrate; alkyl acetates such as triethyl acetate, acetyl triethyl acetate, tributyl acetate, acetyl triethyl acetate, and acetyl tributyl acetate; sucrose fatty acid esters; glycerin mono-, di- and tri-fatty acid esters such as triacetin, glycerin mono-fatty acid esters, glycerin monostearate and acetylated monoglyceride; polyglycerin fatty acid esters; polyethylene glycols such as macrogol 400, macrogol 600, macrogol 1500, macrogol 4000, macrogol 6000, macrogol 20,000, and macrogol 35,000; dibutyl sebacate; tributyl sebacate; vinyl pyrrolidone; propylene glycol; sesame oil; castor oil; glycerin; silicone resins; D-sorbitol; phytosterol; alkyl phthalates such as diethyl phthalate, dibutyl phthalate and dioctyl phthalate; adipate polyesters; isopropyl myristate; medium chain triglyceride; butyl phthalyl butyl glycolate; polyoxyethylene polyoxypropylene glycol; and combinations of any of the foregoing.

Lubricants and anti-adherents may be included in tablet dosage forms provided by the present disclosure to aid in processing. Examples of lubricants and/or anti-adherents useful in tablet dosage forms provided by the present disclosure include calcium stearate, glyceryl behenate, glyceryl monostearate, magnesium stearate, mineral oil, polyethylene glycol, sodium stearyl fumarate, sodium lauryl sulfate, sodium dodecyl sulfate, stearic acid, talc, hydrogenated vegetable oil, zinc stearate, and combinations of any of the foregoing. In certain embodiments, a lubricant is glyceryl monostearate. In certain embodiments, a lubricant is magnesium stearate.

Examples of surfactants useful in tablet dosage forms provided by the present disclosure include pharmaceutically acceptable anionic surfactants, cationic surfactants, zwitterionic, amphoteric (amphiphatic/amphiphilic) surfactants, non-ionic surfactants, polyethyleneglycol esters or ethers, and combinations of any of the foregoing. Examples of useful pharmaceutically acceptable anionic surfactants include monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates such as sodium lauryl sulfate and sodium dodecyl sulfate, ethoxylated alkyl sulfates, ester linked sulfonates such as docusate sodium and dioctyl sodium succinate, alpha olefin sulfonates, or phosphated ethoxylated alcohols. Examples of useful pharmaceutically acceptable cationic surfactants include monoalkyl quaternary ammonium salts, dialkyl quaternary ammonium compounds, amidoamines, and aminimides. Examples of useful pharmaceutically acceptable amphoteric surfactants include N-substituted alkyl amides, N-alkyl betaines, sulfobetaines, and N-alkyl-6-aminopropionates. Examples of useful pharmaceutically acceptable nonioinic surfactants include diblock and triblock copolymers of polyethylene oxide, polypropylene oxide, polyoxyethylene (20) sorbitan monooleate, and polyethyleneglycol esters or ethers such as polyethoxylated castor oil, polyethoxylated hydrogenated castor oil, and hydrogenated castor oil. In certain embodiments, a surfactant is chosen from sodium lauryl sulfate and sodium dodecyl sulfate.

Disintegrants may be included in a tablet formulation to cause a tablet to break apart, for example, by expansion of a disintegrant when exposed to water. Examples of useful disintegrants include water swellable substances such as low-substituted hydroxypropyl cellulose, cross-linked sodium carboxymethylcellulose (sodium croscarmellose), sodium starch glycolate, sodium carboxymethylcellulose, sodium carboxymethyl starch, ion-exchange resins, microcrystalline cellulose, cross-linked polyvinyl pyrrolidone, starches and pregelatinized starch, formalin-casein, alginic acid, certain complex silicates, and combinations of any of the foregoing.

Tablet dosage forms provided by the present disclosure may further comprise one or more coatings, which may partially or fully cover the tablets. While certain coatings may be applied to modify or affect the release of compound (1) from a tablet dosage form in the gastrointestinal tract, others may have no such effect. For example, one or more additional coatings may be for physical protection, aesthetics, ease in swallowing, identification, and/or to facilitate further processing of the tablets. Coatings may be impermeable to moisture or moisture permeable. Moisture impermeable exterior tablet coatings may be useful for maintaining low moisture content in a dosage form that is packaged in the presence of a desiccant and may thereby enhance, for example, the storage stability of a tablet dosage form. Examples of materials useful in coatings for physical protection include permeable or soluble materials such as hydroxypropylmethyl cellulose, hydroxypropyl cellulose, lactose, hydroxypropylethyl cellulose, hydroxyethyl cellulose, and xanthan gum. Examples of materials useful in external tablet coatings to facilitate further processing include talc, colloidal silica, polyvinyl alcohol, titanium dioxide, micronized silica, fumed silica, glycerol monostearate, magnesium trisilicate, and magnesium stearate. An external tablet coating may further include one or more vehicles such as plasticizers, binders, fillers, lubricants, compression aides, and combinations of any of the foregoing. The one or more additional coatings may comprise a single material or a combination of more than one material including any of those disclosed herein. These additional coatings may be applied to tablet dosage forms by methods known to those skilled in the art.

In certain embodiments, dosage forms provided by the present disclosure are substantially free of lactam side products and/or metabolites formed by intramolecular cyclization of compound (1) and/or gabapentin. In certain embodiments, the lactams are metabolites of compound (1) such as 3-azaspiro[4.5]decan-2-one; 3-[3-methyl-1-methylene-5-(methylethyl)hex-5-enyl]-3-azaspiro[4.5]decan-2-one, and 3-[(3-oxo-2-azaspiro[4.5]dec-2-yl)ethyl]-3-azaspiro[4.5]decan-2-one. Dosage forms may be stable to extended storage under normal use conditions, such as for example, greater than one year, without substantial lactam formation such as less than about 0.5 wt-% lactam, less than about 0.2 wt-% lactam, or less than about 0.1 wt-% lactam, where wt-% lactam is determined with respect to the initial amount of compound (1) in the dosage form. In certain embodiments, dosage forms contain less than about 0.2 wt-% lactam following exposure to 40° C./43% relative humidity (RH) for at least 17 days. In certain embodiments, dosage forms contain less than about 2 wt-% lactam and in certain embodiments, less than about 1 wt-% lactam, following exposure to 40° C./75% RH for at least 17 days, where wt-% lactam is determined with respect to the initial amount of compound (1) in the dosage form.

In certain embodiments, an oral dosage form comprises a granulation comprising greater than about 95 wt-% compound (1). In certain embodiments, an oral dosage form comprises a granulation wherein the granulation comprises greater than about 95 wt-% compound (1), may be compressed into a tablet dosage form. In certain embodiments, an oral dosage form comprises a granulation wherein the granulation comprises greater than about 95 wt-% compound (1), may be inserted into and contained in a capsule dosage form. In certain embodiments, an oral dosage form comprising a granulation comprising greater than about 95 wt-% compound (1), may be a liquid oral dosage from such as an emulsion or suspension.

It is generally accepted that commercially acceptable tablets have a friability of less than about 1 wt-% determined according to USP Test No. 1216. In certain embodiments, tablets provided by the present disclosure have a friability of less than about 1 wt-%, in certain embodiments, less than about 0.5 wt-%, in certain embodiments, less than about 0.3 wt-%, and in certain embodiments, less than about 0.2 wt-%.

### Dissolution Profiles of Dosage Forms

The release characteristics of dosage forms provided by the present disclosure comprising compound (1) may be characterized, in part, by the in vitro dissolution profile. Methods for determining dissolution profiles of dosage forms are well known to those skilled in the pharmaceutical arts. Standard methodologies set forth in the U.S. Pharmacopeia may be used. For example, a dissolution profile may be determined using either a U.S. Pharmacopeia Type I Apparatus (baskets) or a U.S. Pharmacopeia Type II Apparatus (paddles).

Using the latter method, dissolution, or release, profiles of dosage forms provided by the present disclosure may be determined by immersing the dosage forms in a 10 mM potassium phosphate monobasic buffer (KH₂PO₄) at pH 7.4, with 1%-vol SLS and a temperature of 37° C. The dissolution medium is stirred at 50 rpm (USP, Type II). Samples are withdrawn from the dissolution medium at intervals and the content of compound (1) in the dissolution medium determined using reverse phase high pressure liquid chromatography (HPLC).

In certain embodiments, release of compound (1) from tablet dosage forms provided by the present disclosure exhibits an in vitro dissolution profile in 10 mM, pH 7.4, potassium phosphate monobasic buffer with 1% sodium lauryl sulfate at 37° C. stirred at 50 rpm (USP, Type II) wherein about 26% to about 41% of the 1-[(α-isobutanoyloxyethoxy)carbonyl]aminomethyl)-1-cyclohexane acetic acid is released at about 4 hours; about 50% to about 78% of the 1-([α-isobutanoyloxyethoxy)carbonyl]aminomethyl)-1-cyclohexane acetic acid is released at about 8 hours; about 68% to about 100% of the 1-([(α-isobutanoyloxyethoxy)carbonyl]aminomethyl)-1-cyclohexane acetic acid is released at about 12 hours; and about 95% to about 100% of the 1-([α-isobutanoyloxyethoxy)carbonyl]aminomethyl)-1-cyclohexane acetic acid is released at about 20 hours.

In certain embodiments, release of compound (1) from tablet dosage forms provided by the present disclosure exhibits an in vitro dissolution profile in 10 mM, pH 7.4, potassium phosphate monobasic buffer with 1% sodium lauryl sulfate at 37° C. stirred at 50 rpm (USP, Type II) wherein about 30% to about 36% of the -([α-isobutanoyloxyethoxy)carbonyl]aminomethyl)-1-cyclohexane acetic acid is released at about 4 hours; about 56% to about 68% of the 1-([α-isobutanoyloxyethoxy)carbonyl]aminomethyl)-1-cyclohexane acetic acid is released at about 8 hours; about 76% to about 94% of the 1-([α-isobutanoyloxyethoxy)carbonyl]aminomethyl)-1-cyclohexane acetic acid is released at about 12 hours; and about 85% to about 100% of the 1-([α-isobutanoyloxyethoxy)carbonyl]aminomethyl)-1-cyclohexane acetic acid is released at about 20 hours.

In certain embodiments, release of compound (1) from tablet dosage forms provided by the present disclosure exhibits an in vitro dissolution profile in 10 mM, pH 7.4, potassium phosphate monobasic (KH₂PO₄) buffer with 1% SLS at 37° C. stirred at 50 rpm (USP, Type II) wherein about 33% of the 1-([α-isobutanoyloxyethoxy)carbonyl]aminomethyl)-1-cyclohexane acetic acid is released at about 4 hours; about 62% of the 1-([(α-isobutanoyloxyethoxy)carbonyl]aminomethyl)-1-cyclohexane acetic acid is released at about 48 hours; about 85% of the 1-([α-isobutanoyloxyethoxy)carbonyl]aminomethyl)-1-cyclohexane acetic acid is released at about 12 hours; and about 95% of the 1-([α-isobutanoyloxyethoxy)carbonyl]aminomethyl)-1-cyclohexane acetic acid is released at about 20 hours.

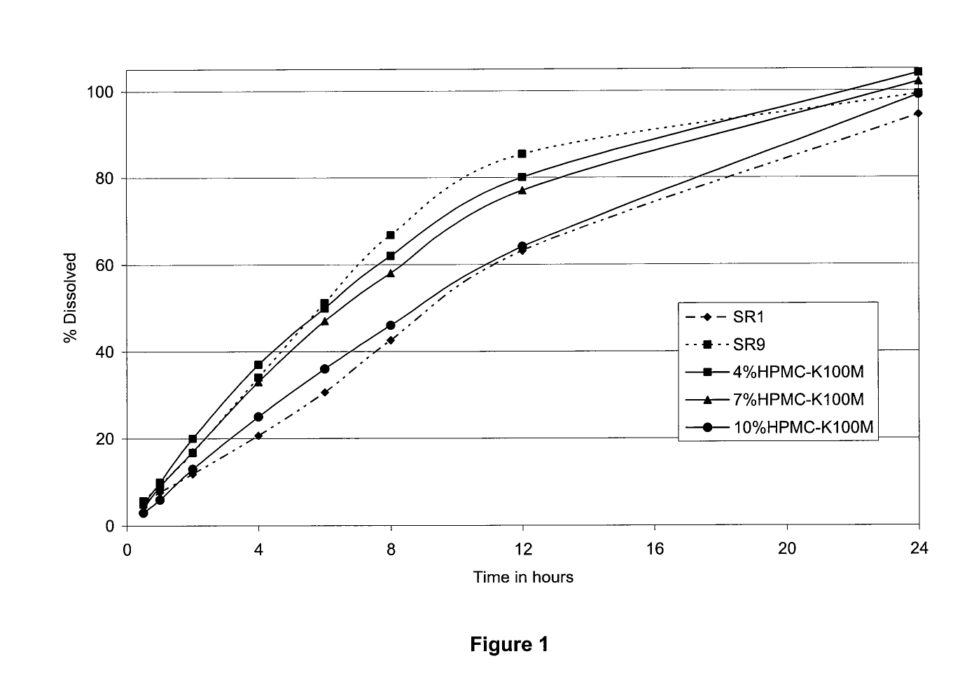

In certain embodiments, a tablet exhibits a dissolution profile that is similar to the foregoing profiles as determined using the f1 difference factor and f2 similarity factor according to FDA guidelines (see Guidance for Industry-Dissolution Testing of Immediate Release Solid Oral Dosage Forms, Center for Drug Evaluation and Research (CDER), August 1997, BP1). In certain embodiments, oral tablet dosage forms provided by the present disclosure exhibit a dissolution profile that when compared with any one of the dissolution profiles shown in FIGS. 1 to 6 produce an f₁ difference factor less than 15 and an f₂ similarity factor from 50 to 100. In certain embodiments, a tablet dosage form comprising from about 80 wt-% to about 95-wt % 1-([α-isobutanoyloxyethoxy)carbonyl]aminomethyl)-1-cyclohexane acetic acid, exhibits a dissolution profile in 10 mM, pH 7.4, potassium phosphate monobasic buffer with 1% sodium lauryl sulfate at 37° C. stirred at 50 rpm (USP, Type II), which when compared with a dissolution profile in which about 33% of the 1-([α-isobutanoyloxyethoxy)carbonyl]aminomethyl)-1-cyclohexane acetic acid is released at about 4 hours; about 62% t of the 1-([α-isobutanoyloxyethoxy)carbonyl]aminomethyl)-1-cyclohexane acetic acid is released at about 8 hours; about 85% of the 1-([α-isobutanoyloxyethoxy)carbonyl]aminomethyl)-1-cyclohexane acetic acid is released at about 12 hours; and about 92% of the 1-([α-isobutanoyloxyethoxy)carbonyl]aminomethyl)-1-cyclohexane acetic acid is released at about 20 hours, provides an f₁ difference factor less than 15 and an f₂ similarity factor from 50 to 100.

In certain of such embodiments, a tablet dosage form exhibiting the foregoing release profiles comprises about 600 mg or 1200 mg compound (1).

### Gabapentin Pharmacokinetics

Sustained release dosage forms comprising compound (1) exhibit enhanced oral bioavailability as gabapentin compared to the oral bioavailability of gabapentin when administered in an equivalent dosage form of gabapentin and/or racemate (Cundy et al., J Pharm Expt'l Ther 2004, 311(1), 315-323; Cundy et al., J Pharm Expt'l Ther 2004, 311(1), 324-333; Cundy et al., 60^{th} American Academy Neurology Annual Meeting, Chicago, Ill., Apr. 12-19, 2008, Poster PO 5.168; Cundy et al., J Clin Pharmacol 2008, 48(12), 1378-88; Lal et al., Clin Therapeutics 2009, 31(8), 1776-1786; and Lal et al., Int'l J Clin Pharm Therapeutics 2010, 48(2), 120-128). The enhanced oral bioavailability of compound (1) is believed to be due the efficient absorption of compound (1) throughout the gastrointestinal tract, including the colon, via passive and/or active transport mechanisms. Dosage forms provided by the present disclosure provide for the release of compound (1) from the dosage form during passage of the dosage form through the gastrointestinal tract.

Following oral administration to a patient, sustained release dosage forms comprising compound (1) provide gabapentin in the systemic circulation of a patient. Compound (1) may be absorbed from the gastrointestinal tract and enter the systemic circulation where the promoiety is cleaved to release gabapentin. The promoiety of compound (1) may be cleaved either chemically and/or enzymatically. For example, one or more enzymes, such as esterases, present in the stomach, intestinal lumen, intestinal tissue, blood, liver, brain, or any other suitable tissue of a mammal can enzymatically cleave the promoiety of compound (1).

When administered orally to a patient, i.e., by a patient swallowing a dosage form provided by the present disclosure, the dosage form provides a sustained therapeutically effective concentration of gabapentin in the blood of the patient during a continuous period of time. In certain embodiments, dosage forms may provide a concentration of gabapentin in the blood of a patient that is greater than a minimum therapeutically effective concentration and less than a minimum adverse concentration of gabapentin in the blood of the patient. In certain embodiments, dosage forms provided by the present disclosure provide a therapeutically effective concentration gabapentin in the blood of a patient for a continuous period of time without exceeding the minimum adverse concentration of gabapentin. In certain embodiments, the concentration of gabapentin in the blood of a patient does not exceed a minimum adverse concentration at any time after the dosage form is orally administered to the patient. Dosage forms provided by the present disclosure can provide a therapeutically effective concentration of gabapentin in the blood of a patient for a continuous period of time while reducing or eliminating adverse drug effects associated with high blood concentrations of gabapentin, e.g., at concentrations above the minimum adverse concentration, observed following oral dosing of forms comprising gabapentin. The high bioavailability of gabapentin achievable using dosage forms comprising compound (1) may facilitate the use of lower mass equivalents of gabapentin in a dose to achieve a sustained therapeutically effective concentration of gabapentin in the blood of a patient compared to the amount of gabapentin in an oral dosage form comprising gabapentin.

Sustained release dosage forms provided by the present disclosure are capable of providing a sustained therapeutically effective concentration of gabapentin in the blood of a patient following oral administration. For example, dosage forms may provide a sustained therapeutically effective concentration of gabapentin in the blood of a patient during a continuous time period selected from at least about 4 hours, at least about 8 hours, at least about 12 hours, at least about 16 hours, at least about 20 hours, or at least about 24 hours, after oral administration to a patient. In certain embodiments, the concentration of gabapentin in the blood of a patient will not exceed a minimum adverse concentration at any time after the dosage form is orally administered to the patient, e.g., will not reach a concentration that causes adverse events in the patient. For certain diseases, therapeutically effective concentration of gabapentin in the blood of a patient may range from about 2 μg/mL to about 12 μg/mL. The pharmacokinetic profile of the blood gabapentin concentration can be characterized by a lower Cₘₐₓ/C₁₂ ratio, and a lower Cₘₐₓ/dose, compared to immediate release and sustained release oral formulations comprising gabapentin. For example, dosage forms may provide a Cₘₐₓ/C₁₂ ratio from about 1.5 to about 3, from about 2.0 to about 2.5, and in certain embodiments about 2.25

In certain embodiments, at least one oral dosage form is administered to a human patient at a dose of compound (1) ranging from about 300 mg to about 1600 mg; and in certain embodiments from about 600 mg to about 1,200 mg; from about 600 to 2,400 mg; from about 800 mg to 3,600 mg; or at a daily dose of compound (1) from about 800 mg to about 7,200 mg, where the weight refers to the amount of 1-([α-isobutanoyloxyethoxy)carbonyl]aminomethyl)-1-cyclohexane acetic acid excluding the weight contribution from any salt and/or solvate thereof. In certain embodiments, at least one oral dosage form is administered to a human patient at a dose of 1-([α-isobutanoyloxyethoxy)carbonyl]aminomethyl)-1-cyclohexane acetic acid ranging from about 300 mg to about 1600 mg, and in certain embodiments from about 600 mg to about 1,200 mg; from about 600 to 2,400 mg, from about 800 mg to 3,600 mg, or at a daily dose of compound (1) from about 800 mg to about 7,200 mg.

A dosage regimen employing oral administration of dosage forms provided by the present disclosure may be developed to maintain a concentration of gabapentin in the blood of a patient, which is greater than a minimum therapeutically effective concentration and less than a minimum adverse concentration for a prolonged period of time. In certain embodiments, a minimum therapeutically effective concentration of gabapentin may range from about 2 μg/mL to about 6 μg/mL. A minimum therapeutic concentration and a minimum adverse concentration will depend on a number of factors such as the disease being treated, the severity of the disease, the intended clinical outcome, the condition of the patient being treated, and so forth. Such regimens may employ repeated dosing of one or more dosage forms provided by the present disclosure. An appropriate interval of dosing may depend, for example, on the amount of compound (1) in the dosage form, the composition of the dosage form, the release characteristics of compound (1) from the dosage form, the disease being treated, the condition of the patient, the potential adverse effects, and the judgment of the prescribing physician. Dosage regimens may include repeated administration of an equivalent dosage form at each interval or different dosage forms at different intervals. For example, a twice-daily dosage regimen can include the administration of a first dosage form in the morning, and a second dosage form in the evening.

Dosage forms provided by the present disclosure further include dosage forms that are bioequivalent to the dosage forms disclosed herein, in terms of both rate and extent of absorption, for example as defined by the U.S. Food and Drug Administration and discussed in “Guidance for Industry-Bioavailability and Bioequivalence Studies for Orally Administered Drug Products” (2003), which is incorporated by reference herein in its entirety.

In certain embodiments, a 1200 mg dose of compound (1) administered to a patient or population of patients, as a sustained release oral dosage form provided by the present disclosure, provides a gabapentin plasma concentration profile characterized by a Cₘₐₓ from about 3.61 μg/mL to about 5.64 μg/mL; a Tₘₐₓ from about 3.92 hour to about 6.12 hour; a T₁₁₂ of about 5.03 hour to about 7.86 hour; and an AUC_{inf} from about 42.3 μg×hr/mL to about 66.1 μg×hr/mL. In certain embodiments, a 1200 mg dose of compound (1) administered to a patient or population of patients, as a sustained release oral dosage form provided by the present disclosure, provides a gabapentin plasma concentration profile characterized by a Cₘₐₓ from about 4.06 μg/mL to about 4.96 μg/mL; a Tₘₐₓ from about 4.41 hour to about 5.39 hour; a T_{1/2} of about 5.66 hour to about 6.92 hour; and an AUC_{inf} from about 47.61 μg×hr/mL to about 58.2 μg×hr/mL. In certain embodiments, a 1200 mg dose of compound (1) administered to a patient or population of patients, as a sustained release oral dosage form provided by the present disclosure, provides a gabapentin plasma concentration profile characterized by a Cₘₐₓ from about 3.61 μg/mL to about 5.64 μg/mL and an AUC_{inf} from about 42.3 μg×hr/mL to about 66.1 μg×hr/mL. In certain embodiments, a 1200 mg dose of compound (1) administered to a patient or population of patients, as a sustained release oral dosage form provided by the present disclosure, provides a gabapentin plasma concentration profile characterized by a Cₘₐₓ from about 4.06 μg/mL to about 4.96 μg/mL and an AUC_{inf} from about 47.61 μg×hr/mL to about 58.2 μg×hr/mL. In certain embodiments, a 1200 mg dose of compound (1) administered to a patient or population of patients, as a sustained release oral dosage form provided by the present disclosure, provides a gabapentin plasma concentration profile characterized by a Cₘₐₓ from about 3.73 μg/mL to about 5.83 μg/mL and an AUC_{inf} from about 43.1 μg×hr/mL to about 67.3 μg×hr/mL. The 1200 mg dose of compound (1) may be administered as a single 1200 mg dosage form, or as two, 600 mg dosage forms. In certain of the preceding embodiments, gabapentin plasma concentration profile represents a mean for a population of patients consisting of ten (10) healthy adult male patients under fasted conditions.

In certain embodiments, dosage forms provided by the present disclosure when administered at an oral dose of 1200 mg 1-([α-isobutanoyloxyethoxy)carbonyl]aminomethyl)-1-cyclohexane acetic acid provides a plasma gabapentin between about 2 μm/mL and about 6 μgm/mL for at least about 6 hours, for at least about 10 hours, and in certain embodiments, at least about 15 hours.

### Therapeutic Uses

Sustained release oral dosage forms provided by the present disclosure may be administered to a patient suffering from any disease or disorder for which the parent drug, gabapentin, is known, believed to be, or hereafter determined to be therapeutically effective. Indications for which gabapentin has been prescribed, and hence for which the dosage forms provided by the present disclosure are also effective, include epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, or vulvodynia. In certain embodiments, oral tablet dosage forms provided by the present disclosure may be used to treat restless legs syndrome. In certain embodiments, oral tablet dosage forms provided by the present disclosure may be used to treat neuropathic pain, and in certain embodiments, post-herpetic neuralgia or painful diabetic neuropathy.

The suitability of dosage forms provided by the present disclosure in treating the above-listed diseases may be determined by methods described in the art.

A suitable dose of compound (1) to be administered to a patient in need of gabapentin therapy may be estimated based on the mass equivalent of gabapentin and the enhanced oral bioavailability of gabapentin provided by compound (1).

In certain embodiments, oral tablet dosage forms provided by the present disclosure may be used to treat epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, or vulvodynia. In certain embodiments, oral tablet dosage forms provided by the present disclosure may be used to treat restless legs syndrome. In certain embodiments, oral tablet dosage forms provided by the present disclosure may be used to treat neuropathic pain such as post-herpetic neuralgia or painful diabetic neuropathy.

In certain embodiments, oral tablet dosage form provided by the present disclosure may be used for prophylaxis of a disease is chosen from epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, and vulvodynia. In certain embodiments, oral tablet dosage forms provided by the present disclosure may be used for the prophylaxis of restless legs syndrome. In certain embodiments, oral tablet dosage forms provided by the present disclosure may be used for the prophylaxis of neuropathic pain such as post-herpetic neuralgia or painful diabetic neuropathy.

### Dosing

It is believed that tablet dosage forms providing sustained systemic concentrations of gabapentin will enhance patient compliance as compared to the non-prodrug form which is currently administered up to six times per day, a regimen that is inconvenient for patients and difficult for patients to remember. Additionally, it is believed that the use of tablet oral dosage forms provided by the present disclosure will provide enhanced efficacy with reduced side effects which side effects may include dizziness, somnolence, fatigue and/or ataxia.

The amount of compound (1) that will be effective in the treatment of a particular disease disclosed herein will depend, at least in part, on the nature of the disease, and may be determined by standard clinical techniques known in the art. In addition, in vitro or in vivo assays may be employed to help identify optimal dosing ranges. Dosing regimens and dosing intervals may also be determined by methods known to those skilled in the art. The amount of compound (1) administered may depend on, among other factors, the subject being treated, the weight of the subject, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of compound (1) can be adjusted to provide an equivalent molar quantity or mass equivalent dose of gabapentin. A dose can comprise multiple dosage forms provided by the present disclosure. Therapeutically effective doses of gabapentin in pediatric patients are from about 25 mg to about 50 mg per kilogram body weight per day. In certain embodiments, for adult patients, a daily dose can comprise a mass equivalent of gabapentin ranging from about 100 mg to about 3,600 mg, in certain embodiments, from about 300 mg to about 3,600 mg, in certain embodiments, from about 600 mg to about 2,400 mg, and in certain embodiments, from about 600 mg to about 1,200 mg. The dose of compound (1) and appropriate dosing intervals can be selected to maintain a sustained therapeutically effective concentration of gabapentin in the blood of a patient, and in certain embodiments, without exceeding a minimum adverse concentration.

In certain embodiments, dosage forms provided by the present disclosure may be administered once per day, twice per day, and in certain embodiments at intervals of more than once per day. Dosing may be provided alone or in combination with other drugs and may continue as long as required for effective treatment of the disease. Dosing includes administering a dosage form to a mammal, such as a human, in a fed or fasted state.

A dose may be administered in a single dosage form or in multiple dosage forms. When multiple dosage forms are used the amount of compound (1) contained within each of the multiple dosage forms may be the same or different.

During treatment a dose and dosing schedule may provide sufficient or steady state systemic concentration of gabapentin to treat a disease. In certain embodiments, an escalating dose may be administered.

### Combination Therapy

Dosage forms provided by the present disclosure may further comprise one or more pharmaceutically active compounds in addition to compound (1). Such compounds may be provided to treat the same disease or a different disease than the disease being treated with compound (1).

In certain embodiments, compound (1) may be used in combination with at least one other therapeutic agent. In certain embodiments, compound (1) may be administered to a patient together with another compound for treating epilepsy, essential tremor, chronic regional pain syndrome, fibromyalgia, radiculopathy, abdominal-visceral pain, irritable bowel syndrome, migraine, generalized anxiety disorder, depression, insomnia, over active bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, or vulvodynia. In certain embodiments, the at least one other therapeutic agent may be a different gabapentin prodrug. Compound (1) and the at least one other therapeutic agent may act additively or, and in certain embodiments, synergistically. The at least one additional therapeutic agent may be included in the same dosage form comprising compound (1) or may be in a separate dosage form. Accordingly, methods provided by the present disclosure can further include, in addition to administering compound (1), administering one or more therapeutic agents effective for treating the same or different disease than the disease being treated by compound (1). Methods provided by the present disclosure include administration of compound (1) and one or more other therapeutic agents provided that the combined administration does not inhibit the therapeutic efficacy of compound (1) and/or does not produce adverse combination effects.

In certain embodiments, dosage forms comprising compound (1) may be administered concurrently with the administration of another therapeutic agent, which may be part of the same dosage form as, or in a different dosage form than that comprising compound (1). Compound (1) may be administered prior or subsequent to administration of another therapeutic agent. In certain embodiments of combination therapy, the combination therapy may comprise alternating between administering compound (1) and a composition comprising another therapeutic agent, e.g., to minimize adverse drug effects associated with a particular drug. When compound (1) is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising compound (1) may be administered with one or more substances to enhance, modulate and/or control release, bioavailability, therapeutic efficacy, therapeutic potency, stability, and the like of compound (1). For example, to enhance the therapeutic efficacy of compound (1) or its metabolite, gabapentin, compound (1) may be co-administered with or a dosage form comprising compound (1) may comprise one or more active agents to increase the absorption or diffusion of compound (1) or gabapentin from the gastrointestinal tract to the systemic circulation, or to inhibit degradation of compound (1) or gabapentin in the blood of a patient. In certain embodiments, a dosage form comprising compound (1) may be co-administered with an active agent having pharmacological affects that enhance the therapeutic efficacy of compound (1).

Additionally, dosage forms provided by the present disclosure may be used in combination with other drugs that are themselves known to cause epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, and vulvodynia as an adverse effect, thereby preventing or reducing the occurrence of such adverse effects.

### EXAMPLES

The following examples describe in detail the preparation and properties of tablet dosage forms comprising compound (1). It will be apparent to those skilled in the art that many modifications, both to materials and methods, may be practiced without departing from the scope of the disclosure.

### Example 1

### {[(1-Isobutanoyloxyethoxy)carbonyl]aminomethyl}-1-cyclohexane Acetic Acid (1)

{[(1-Isobutanoyloxyethoxy)carbonyl]aminomethyl}-1-cyclohexane acetic acid (1) may be prepared using any of the methods disclosed in Gallop et al., U.S. Pat. Nos. 6,818,787, 7,186,855, 7,227,028, and 6,927,036; Estrada et al., US 2005-0154057; Bhat et al., US 2005-0070715; Raillard et al., U.S. Pat. No. 7,332,924, and U.S. application Ser. Nos. 12/537,764 and 12/537,798. For example,

### O-(1-Chloroethyl) S-methyl thiocarbonate (1a)

A solution of methanethiol (170 g, 3.5 mol) and 1-chloroethyl chloroformate (386 mL, 502 g, 3.5 mol) in CH₂Cl₂ (1 L) was cooled to 0° C. in an ice-water bath. N-Methylmorpholine (388 mL, 357 g, 3.53 mol) was added dropwise over a period of 1 h and the reaction mixture was allowed to stir at room temperature for 16 h. The reaction mixture was diluted with CH₂Cl₂ (2 L), washed with water (1 L), saturated bicarbonate solution (1 L) and brine (1 L), then dried over anhydrous sodium sulfate, filtered and concentrated in vacuo. The residue was purified by vacuum distillation (95° C./20 Torr) to provide the title compound (1a) as a colorless liquid (510 g, 94% yield). ¹H NMR (CDCl₃, 400 MHz): δ 1.82 (d, J=5.6 Hz, 3H), 2.38 (s, 3H), 6.57 (q, J=5.2 Hz, 1H).

### O-(1-Isobutanoyloxyethyl) S-methyl thiocarbonate (1b)

Compound (1a) (308 mg, 2 mmol) was dissolved in isobutyric acid (264 mg, 3 mmol). This mixture was slowly added to a pre-mixed solution of isobutyric acid (264 mg, 3 mmol) and diisopropylethylamine (387 mg, 3 mmol) and the reaction mixture heated to 55° C. for 16 h, diluted with ether (50 mL), washed with water (2×10 mL), saturated bicarbonate solution (2×10 mL) and brine (10 mL), then dried over anhydrous sodium sulfate, filtered and concentrated in vacuo to give the title compound (1b) as a colorless liquid (400 mg, 97%). The product was further purified by vacuum distillation (135° C./20 Torr). ¹H NMR (CDCl₃, 400 MHz): δ 1.17 (d, J=6.8 Hz, 6H), 1.49 (d, J=5.6 Hz, 3H), 2.33 (s, 3H), 2.54 (m, 1H), 6.91 (q, J=5.2 Hz, 1H).

### [(1-Isobutanoyloxyethoxy)carbonyloxy]succinimide (1c)

To a solution of compound (1b) (1 g, 4.8 mmol) in CH₂Cl₂ (10 mL) was added N-hydroxysuccinimide (1.1 g, 9.5 mmol) and the reaction mixture cooled to 0° C. A solution of 32% (v/v) peracetic acid in acetic acid (3.4 mL, 1.1 g, 14.4 mmol) was added dropwise over a period of 10 min, then the solution allowed to stir at room temperature for 3 h. The reaction mixture was diluted with ether (50 mL) and washed with water (2×10 mL), saturated sodium bicarbonate solution (10 mL) and brine (10 mL), then dried over anhydrous sodium sulfate, filtered and concentrated in vacuo to give the title compound (1c) as a colorless oil (1 g, 77%). After trituration with hexane (20 mL) the product solidified to a white solid. m.p: 50-54° C. ¹H NMR (CDCl₃, 400 MHz): δ 1.17 (d, J=6.8 Hz, 6H), 1.56 (d, J=5.6 Hz, 3H), 2.55 (m, 1H), 2.82 (s, 4H), 6.80 (q, J=5.2 Hz, 1H). MS (ESI) m/z 296.4 (M+Na)⁺.

### {[(1-Isobutanoyloxyethoxy)carbonyl]aminomethyl}-1-cyclohexane acetic acid (1)

To a solution of gabapentin (1.7 g, 10 mmol) and sodium bicarbonate (20 mmol) in water (40 mL) was added a solution of compound (1c) (2.73 g, 10 mmol) in acetonitrile (20 mL) over 1 min. The reaction was stirred at room temperature for 16 h. The reaction mixture was diluted with diethyl ether (100 mL) and washed with 0.1 M aqueous potassium bisulfate (3×100 mL). The organic phase was separated, dried over anhydrous magnesium sulfate, filtered, and concentrated in vacuo to afford the title compound (1) as a white solid (2.7 g, 96%). The product was recrystallized by dissolution in 1:10 ethyl acetate:heptane (10 mL) at 60° C., followed by slow cooling to 4° C. The white crystalline product was isolated by filtration. Melting point: 63-64° C. ¹H NMR (CDCl₃, 400 MHz): δ 1.15 (d, 6H), 1.40-1.55 (m, 10H), 1.45 (d, 3H), 2.32 (s, 2H), 2.49-2.56 (m, 1H), 3.23 (d, 2H), 5.41 (t, 1H), 6.75 (q, 1H). MS (ESI) m/z 330.29 (M+H⁺).

{[(1-Isobutanoyloxyethoxy)carbonyl]aminomethyl}-1-cyclohexane acetic acid (1) may be crystallized using the procedures described by Estrada et al., US 2005-0154057. For example,

### Crystallization of {[(1-isobutanoyloxyethoxy)carbonyl]aminomethyl}-1-cyclohexane acetic acid

Compound (1) (12 g) was suspended in methylcyclohexane: methyl tert-butyl ether 10:1 (60 mL). The suspension was slowly heated up to 50° C. over a period of 30 minutes. The clear solution was then allowed to cool to room temperature. The turbid mixture was seeded with 5 mg of the title compound in crystalline form. The mixture was further cooled to 0-4° C. for 2 hours. The solid product was filtered and washed with methylcyclohexane (2×10 mL) to yield crystalline compound (1) as a white crystalline solid (10 g, 83% yield). The crystalline solid material had a melting point of about 64-66° C. as measured by open capillary melting point determination.

### Example 2

### Flow Characterization of Dry Powders

The flow of dry powders was characterized using a FLODEX™ Powder Flowability Index Test Instrument (Hanson Research Corporation, Chatsworth, Calif.). The instrument was equipped with a cylindrical metal reservoir, which holds the test powder prior to flow testing. The cylindrical reservoir has an inside diameter of 5.7 cm and a length of 7.4 mm. The bottom end of the reservoir can be closed with removable metal discs. Each disc has a round orifice centered in the disc. Orifice diameters range from 4 mm to 10 mm in 1 mm increments, and from 10 mm to 34 mm in 2 mm increments. Prior to flow testing, the orifice is blocked. Powder is then placed over the blocked orifice. When the orifice is unblocked, powder can flow through the orifice under the force of gravity if the orifice diameter is sufficiently large. Powder that flows through small orifices is considered to have flow properties useful for tableting. For example, a Flodex measurement (Flodex) of less than about 24 mm is typically used for high-speed tableting operations at commercial scale. A Flodex less than about 20 mm is useful for high-speed tableting operations. A Flodex of 18 mm or less is considered especially useful for high throughput tableting operations.

The Flodex is determined by first gently filling the reservoir while the orifice at the bottom is blocked with approximately 70 cc of test powder, while avoiding severe piling, and without vibrating or tapping the powder bed. Next, the orifice is unblocked. This can be accomplished by opening a shutter that is supplied with the instrument. Alternatively, if a shutter is not used, the powder-filled reservoir fitted with a disc can be set on a dry, flat surface to block the orifice. Then, slowly and evenly, the reservoir is lifted to allow the powders to flow. In either procedure, if the powder flows through the orifice, a clear channel is left within the powder bed. If the powder does not flow through the orifice, an arch-shaped cavity within the powder bed is formed above the orifice and is referred to as an arch. The flow test is conducted with various orifice sizes until the minimum orifice size for good flow is identified, which is referred to as the Flodex. The Flodex is the minimum orifice diameter at which the powder flows through the orifice more times than it does not in at least three measurement trials.

### Example 3

### Dissolution Profiles of Tablet Formulations

Dissolution profiles for tablets were obtained using a USP paddle apparatus (Type II) in 900 mL of 10 mM potassium phosphate monobasic (KH₂PO₄) buffer at pH 7.4, with 1%-vol sodium lauryl sulfate (SLS) at a temperature of 37° C. The paddle stirring speed was 50 rpm.

### Example 4

### Granulation and Tableting

Compound (1), sodium lauryl sulfate (SLS), and hydroxypropylmethyl cellulose (METHOCEL™-E4M, Dow Chemical) were weighed and sieved through a 30-mesh screen to break up soft agglomerates. The screened materials were placed into a high shear wet granulator (KG-5 high sheer blender, 5 L bowl, Key International) and pre-blended for 5 min (impeller speed of 188 rpm and chopper speed of 2000 rpm). Water was weighed out (USP, 27.5 wt-%), added to the granulator over ca. 20 minutes, and the wet mass blended for 2 min at an impeller speed of 188 rpm and a chopper speed of 2,000 rpm. After granulation the wet granules were milled through a Quadro Comil fitted with a 0.079G screen. The milled wet granules were placed in a dryer (GPCG-2 Fluid Bed Dryer, Glatt GmbH) fitted with a 6 L bowl and dried at an inlet temperature of 55° C. and airflow adjusted at 35 SCFM until the granule temperature began to rise, i.e., <28° C. The dried granules were milled by passing the granules through the Quadro Comil fitted with a 0.050G screen.

To prepare the tableting formulation, the dried granules were transferred to a V-shell, 0.5-quart blender and hydroxypropylmethyl cellulose (METHOCEL™ K100M, Dow Chemical), previously passed through a 30-mesh screen, was added and mixed for 5 min at 25 rpm. Magnesium stearate (NF, non-bovine, Mallinckrodt) was sieved through a 70-mesh screen, added to the blender, and the contents mixed for 3 min at 25 rpm. Tablets were prepared from the blend using a Korsch XL100 tableting press equipped with D- or B-tooling.

The composition of granules and tablets disclosed in this example are summarized in Table 1 and Table 2.

TABLE 1
Granule composition.

| Component | wt-% |
|---|---|
| Compound (1) | 98.0 |
| SLS | 1.0 |
| METHOCEL ™-E4M | 1.0 |
| Water | 27.5 |

TABLE 2
Composition of 600 mg and 1200 mg tablets.

| | |
|---|---|
| 600 mg tablet | 1200 mg tablet |

| Component | wt-% | mg | wt-% | mg |
|---|---|---|---|---|
| Granulation | 89.0¹ | 534 | 91.0² | 1080 |
| METHOCEL ™- | 8.0 | 48 | 7.0 | 84 |
| K100M | | | | |
| Magnesium stearate | 3.0 | 18 | 2.0 | 36 |
| Total | 100.0 | 600 | 100.0 | 1200 |

¹Corresponding to 87.22 wt-% compound (1); 0.89 wt-% SLS; and 0.89 wt-% METHOCEL ™-E4M.
²Corresponding to 89.2 wt-% compound (1); 0.90 wt-% SLS; and 0.90 wt-% METHOCEL ™-E4M.

### Example 5

### Preparation of Tablets by Roller Compaction

Sustained release tablets containing 600 mg compound (1) were prepared using roller compaction. The tablet components are shown in Table 3. Tablets were prepared using standard roller compaction processing. Compound (1) and a portion of the dibasic calcium phosphate dihydrate were pre-blended in a diffusion mixer. The pre-blend, talc, glyceryl behenate, colloidal silicon dioxide, and the remaining dibasic calcium phosphate dihydrate were passed through a screening mill, and then combined with a portion of the sodium lauryl sulfate and a portion of pre-screened magnesium stearate, and blended in the diffusion mixer. The blend was roller compacted to provide a dry granulation, which was then blended with the remaining portion of sodium lauryl sulfate and the remaining portion of pre-screened magnesium stearate. The blend was then compressed into tablets. Tablets prepared by roller compaction contained about 46 wt-% compound (1). The composition of the SR1 and SR9 tablet formulations was similar except that compound (1) from different synthetic lots was used.

TABLE 3
Composition of 600 mg compound (1) SR tablets prepared using roller
compaction.

| Component | SR1 | SR9 |
|---|---|---|
| Compound (1) (wt-%) | 45.80 | 45.80 |
| Colloidal silicon dioxide (wt-%) | 0.41 | 0.41 |
| Dibasic calcium phosphate (wt-%) | 39.56 | 39.56 |
| Glyceryl behenate (wt-%) | 4.59 | 4.59 |
| Sodium lauryl sulfate (wt-%) | 1.83 | 1.83 |
| Talc (wt-%) | 6.11 | 6.11 |
| Magnesium stearate (wt-%) | 1.70 | 1.70 |
| Tablet weight (mg) | 1310 | 1310 |

### Example 6

### Effect of Release Rate-Controlling Polymer on Tablet Dissolution

To assess the effect of wt-% METHOCELT™-K100M on the 1200 mg tablet dissolution profile, tablets comprising 4, 7, or 10 weight percent METHOCELT™-K100M prepared according to Example 4 using modified oval (0.8350 mm×0.3225 mm) tooling. The compositions of the corresponding tablet formulations are shown in Table 4. The dissolution profiles are shown in FIG. 1 and compared with the dissolution profiles for SR1 and SR9 tablets prepared according to Example 5.

TABLE 4
Composition of 1200 mg compound (1) tablets having different
weight percent METHOCEL ™-K100M.

| | Blend A | Blend B | Blend C |
|---|---|---|---|
| Component | wt-% | wt-% | wt-% |
| Compound (1) | 92.1 | 89.2 | 87.2 |
| METHOCEL ™-E4M | 0.9 | 0.9 | 0.9 |
| SLS | 0.9 | 0.9 | 0.9 |
| METHOCEL ™- | 4.0 | 7.0 | 10.0 |
| K100M | | | |
| Magnesium Stearate | 2.0 | 2.0 | 2.0 |
| Total | 100 | 100 | 100 |
| Tablet weight (mg) | 1303 | 1345 | 1392 |

### Example 7

### Effect of HPMC Type and Content on Tablet Dissolution

To determine the impact of HPMC type and content on tablet dissolution, 600 mg dose tablets were prepared using granules containing 98 wt-% compound (1), 1 wt-% METHOCELT™-E4M, and 1 wt-% SLS and prepared using 20 wt-% water; and either METHOCEL™-K100M or METHOCELT™-E4M. The content of the tablets in wt-% is shown in Table 5.

TABLE 5
Content of tablets containing different amounts of METHOCEL ™-K100M or
METHOCEL ™-K4M.

| | Component | | | | | | | | |
|---|---|---|---|---|---|---|---|---|---|
| | L7 | L8 | L9 | L10 | L11 | L12 | L13 | L14 | L15 |
| Granules (wt-%) | 92 | 89 | 86 | 83 | 92 | 89 | 86 | 83 | 87 |
| METHOCEL ™- | 0 | 0 | 0 | 0 | 6 | 9 | 12 | 15 | 11 |
| K100M (wt-%) | | | | | | | | | |
| METHOCEL ™- | 6 | 9 | 12 | 15 | 0 | 0 | 0 | 0 | 0 |
| E4M (wt-%) | | | | | | | | | |
| Magnesium | 2 | 2 | 2 | 2 | 2 | 2 | 2 | 2 | 2 |
| Stearate (wt-%) | | | | | | | | | |
| Total (wt-%) | 100 | 100 | 100 | 100 | 100 | 100 | 100 | 100 | 100 |
| Scale (g) | 17.4 | 18.0 | 18.6 | 19.3 | 17.4 | 18.0 | 18.6 | 19.3 | 20 |
| Hardness (kP) | 17.3 | 17.8 | 19.0 | 19.2 | 18.2 | 18.1 | 16.2 | 21.8 | 19.0 |
| Thickness (mm) | 6.40 | 6.47 | 6.73 | 6.97 | 6.39 | 6.53 | 6.76 | 6.98 | 6.62 |

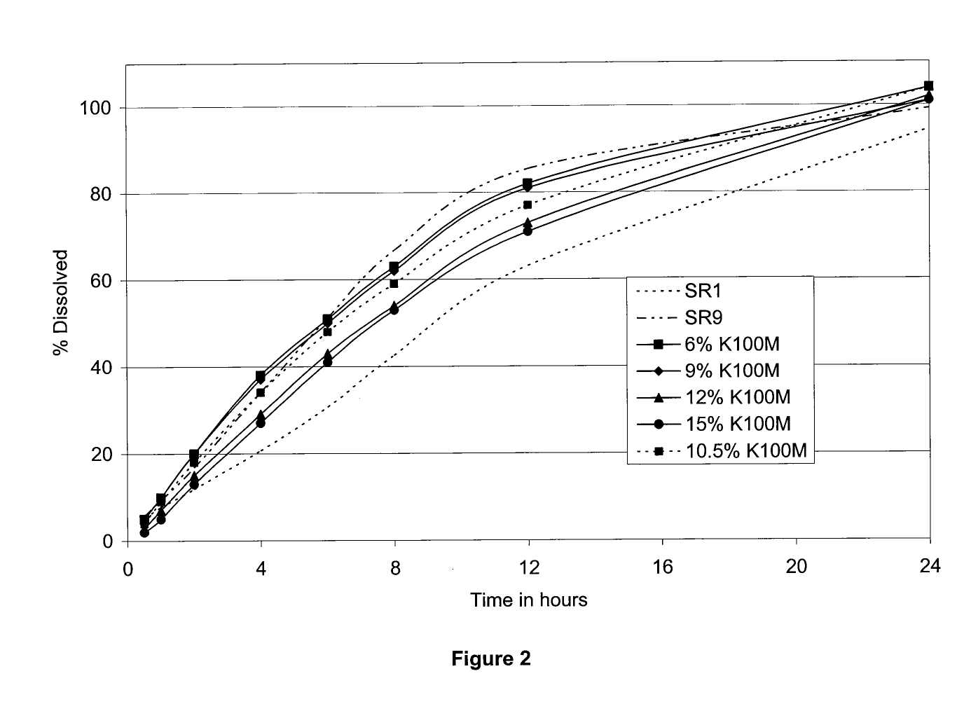

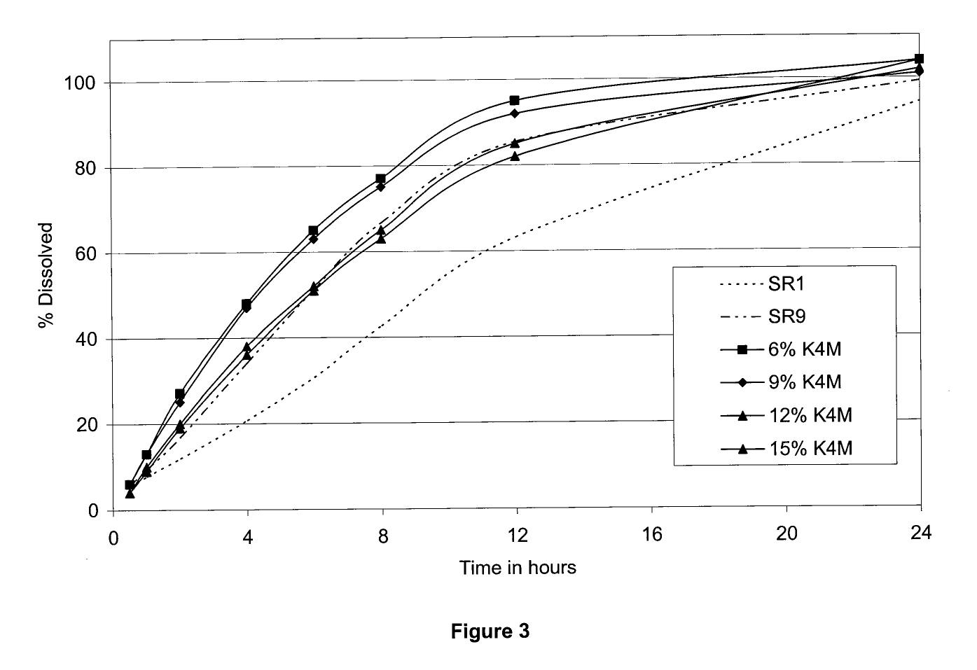

Dissolution profiles of tablets prepared from formulations blended with either METHOCELT™-K100M or METHOCEL™-K4M are shown in FIG. 2 and FIG. 3, respectively.

### Example 8

### Effect of Tablet Size on Dissolution

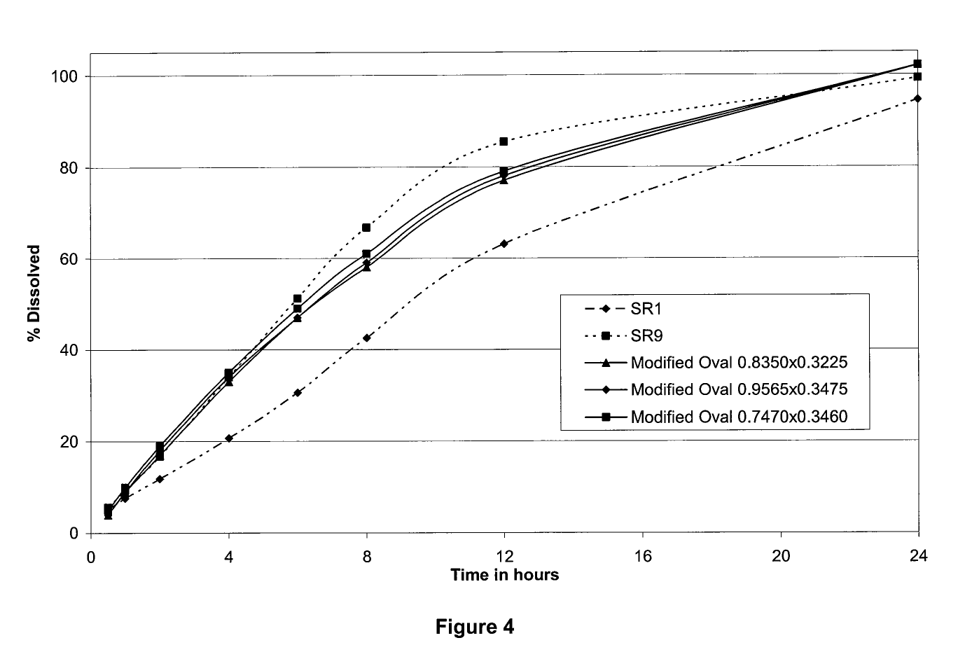

The effect of tablet size on the dissolution profile was assessed by comparing the dissolution of tablets having a total weight of about 1350 mg and containing 89 wt-% compound (1) (1200 mg compound (1)), 7 wt-% METHOCELT™-K100M, and 2 wt-% magnesium stearate and fabricated using modified oval tooling of different dimensions. The dissolution profiles for tablets fabricated using (0.8350 mm×0.3225 mm), (0.9565 mm×0.33475 mm) and (0.7470 mm×0.3460 mm) modified oval tooling are shown in FIG. 4.

### Example 9

### Effect of Tablet Hardness on Dissolution

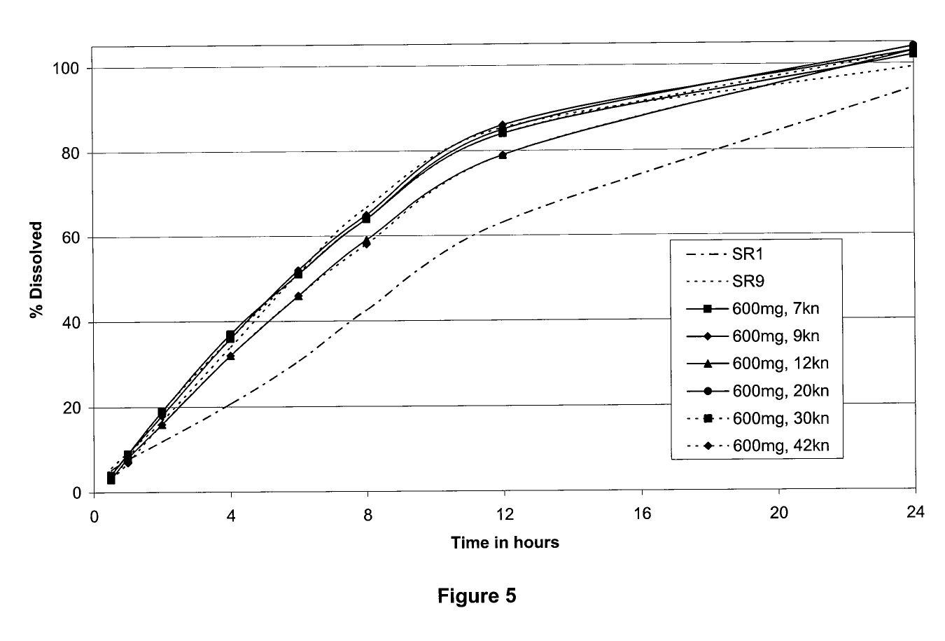

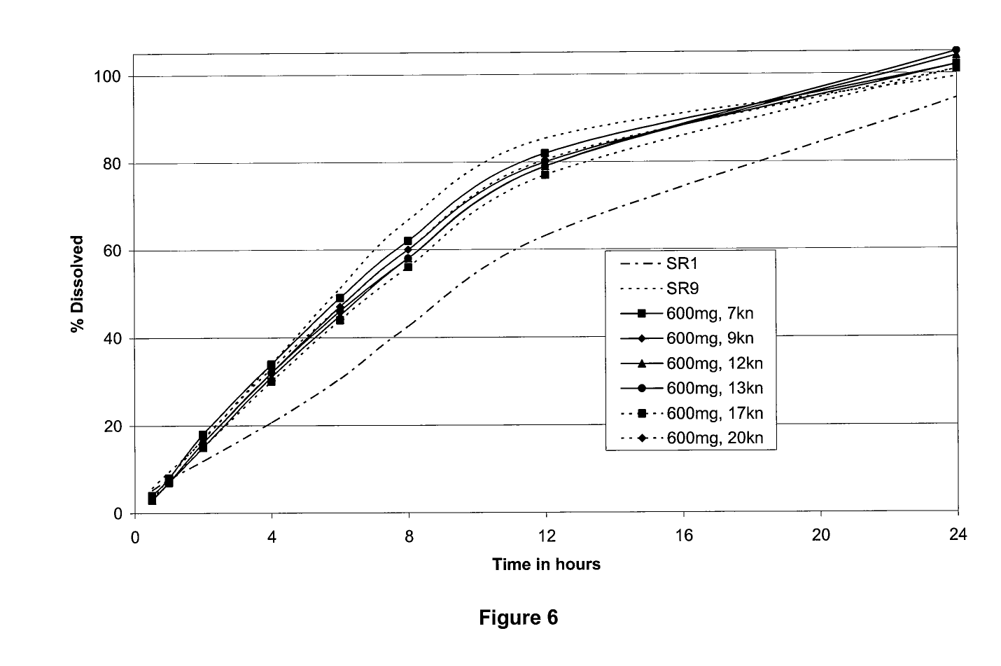

The effect of tablet hardness on the dissolution of tablets containing 2 wt-% magnesium stearate (88.2 wt-% compound (1), 0.9 wt-% METHOCEL™-E4M, 0.9 wt-% SLS, 8.0 wt-% METHOCELT™-K100M, and 2 wt-% magnesium stearate; total weight 680.3 mg) or 3 wt-% magnesium stearate (87.2 wt-% compound (1), 0.9 wt-% METHOCEL™-E4M, 0.9 wt-% SLS, 8.0 wt-% METHOCEL™-K100M, and 3 wt-% magnesium stearate; total weight 687.9 mg) is shown in FIG. 5 and FIG. 6, respectively. For tablets comprising 2 wt-% magnesium stearate (FIG. 5), the compression forces of 7, 9, 12, 20, 30, and 42 kN, correspond to tablet hardness of 12.7, 14.4, 17.9, 16.8, 17.3, and 15.8 kP, respectively. For tablets comprising 2 wt-% magnesium stearate (FIG. 6), the compression forces of 7, 9, 12, 20, 30, and 42 kN, correspond to tablet hardness of 12.7, 14.4, 17.9, 16.8, 17.3, and 15.8 kP, respectively.

### Example 10

### Effect of Water Content During Granulation on Granule Properties

The impact of the amount of water used during high shear wet granulation on the strength of granules was determined. Granulations containing either 98 wt-% compound (1), 1 wt-% METHOCELT™-E4M and 1 wt-% SLS, or 97 wt-% compound (1) and 3 wt-% METHOCEL™-E4M, were prepared using from 20 wt-% to 30 wt-% water, dried, and the granule strength measured by determining the air pressure at which the particles began to fracture using a Sympatec particle analyzer (QicPic/RODOS-L/VIBRI-L, Sympatec GmbH, Clausthal-Zellerfeld, DE).

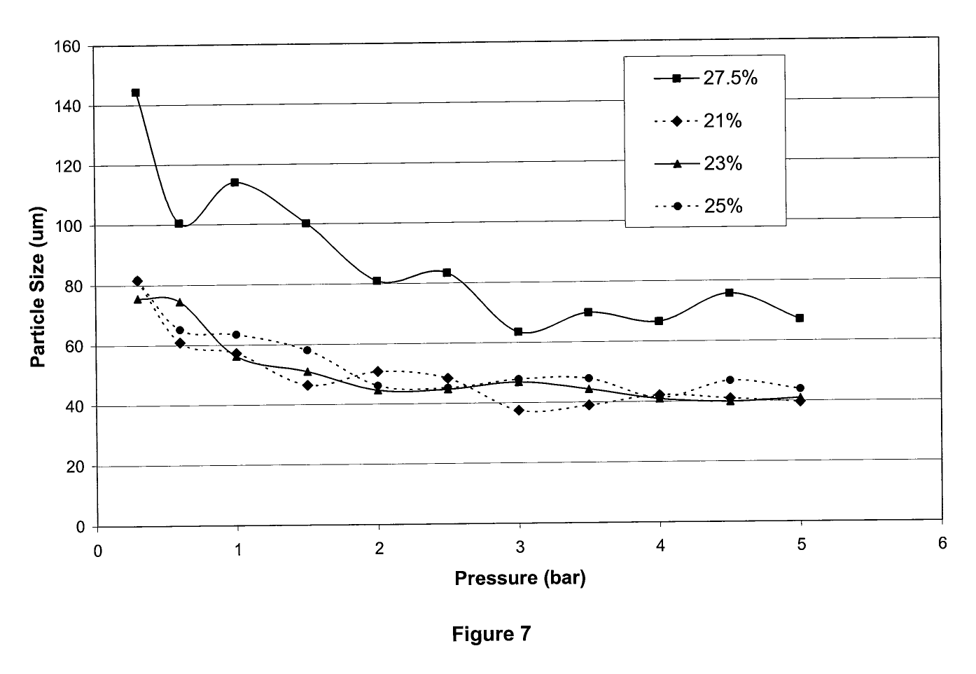

The mean particle size of d10 fraction granules containing 97 wt-% compound (1) and 3 wt-% METHOCELT™-E4M prepared using different amounts of water with increasing air pressure is shown in FIG. 7.

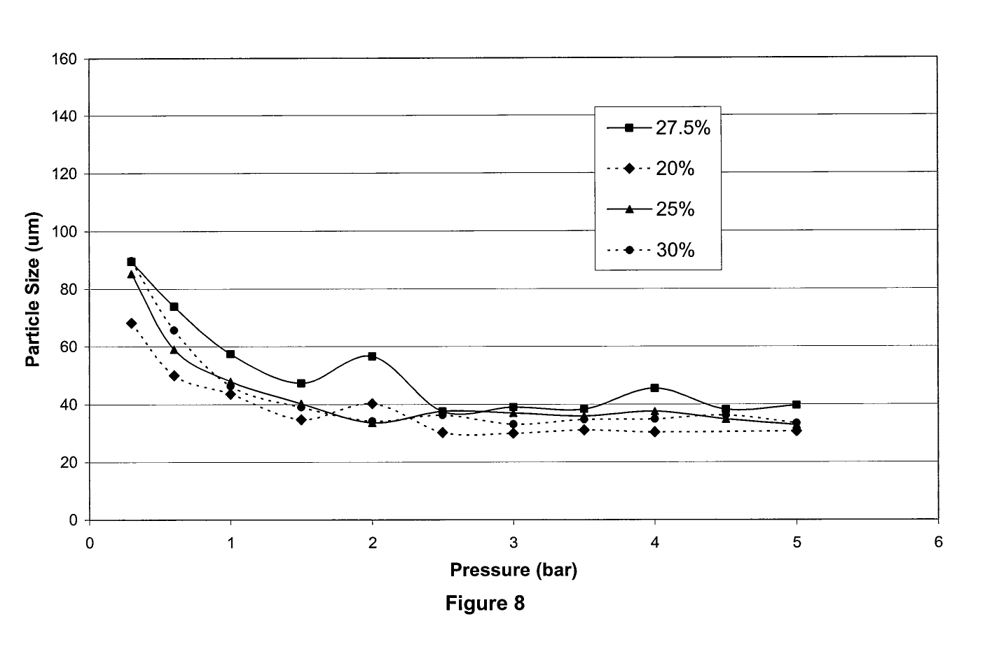

The mean particle size of d10 fraction granules containing 97 wt-% compound (1), 1 wt-% METHOCEL™-E4M, and 1 wt-% SLS and prepared using from 20 wt-% to 30 wt-% water with increasing air pressure is shown in FIG. 8.

The effect of water on the flow properties of the granulations prepared using different amounts of water are shown in Table 6. Granulations having 98 wt-% compound (1), 1 wt-% METHOCELT™-E4M, and 1 wt-% SLS exhibited acceptable flow properties over a wide range of water used in the granulation process.

TABLE 6
Flow properties of granulations prepared using different amounts of water.

| | | Bulk | Tap | | | |
|---|---|---|---|---|---|---|
| | | Density | Density | Carr | Hausner | Flodex |
| | Water (wt-%) | (gm/mL) | (gm/mL) | Index | Ratio | (mm) |
| Compound (1) | — | 0.329 | 0.450 | 27.0 | 1.37 | >34 |
| 98% cmpd (1) | 20 | 0.416 | 0.507 | 18.0 | 1.22 | 16 |
| 1 wt-% | 25 | 0.432 | 0.508 | 15.0 | 1.18 | 7 |
| METHOCEL ™-E4M | 27.5 | 0.485 | 0.561 | 13.4 | 1.15 | 8 |
| 1 wt-% SLS | 30 | 0.430 | 0.505 | 15.0 | 1.18 | 8 |
| 97% cmpd (1) | 21 | 0.410 | 0.518 | 21.0 | 1.27 | 16 |
| 3 wt-% | 23 | 0.435 | 0.53 | 18.0 | 1.22 | 20 |
| METHOCEL ™- | 25 | 0.418 | 0.509 | 18.0 | 1.22 | 13 |
| E4M | 27.5 | 0.407 | 0.492 | 17.3 | 1.21 | 7 |

### Example 11

### Properties of Tablet Components and Formulations

Flow properties of tablet components and formulations are shown in Table 7. The Main Blend contained 98 wt-% compound (1), 1 wt-% METHOCELT™-E4M, and 1 wt-% SLS. Blend D contained 90 wt-% granules blended with 8 wt-% METHOCEL™-K100M and 2 wt-% magnesium stearate, and Blend E contained 89 wt-% granules blended with 8 wt-% METHOCEL™-K100M and 3 wt-% magnesium stearate and were prepared as described in Example 4.

TABLE 7
Flow properties of tablet components and formulations.

| | Initial | Tap | | | |
|---|---|---|---|---|---|
| | Density | Density | Carr | Hausner | Flodex |
| Material | (gm/mL) | (gm/mL) | Index | Ratio | (mm) |
| Main Blend before | 0.280 | 0.444 | 37.0 | 1.59 | 34 |
| granulation | | | | | |
| Granules | 0.475 | 0.546 | 12.0 | 1.14 | 10 |
| Blend D | 0.469 | 0.565 | 17.0 | 1.21 | 17 |
| Blend E | 0.463 | 0.563 | 17.8 | 1.22 | 20 |

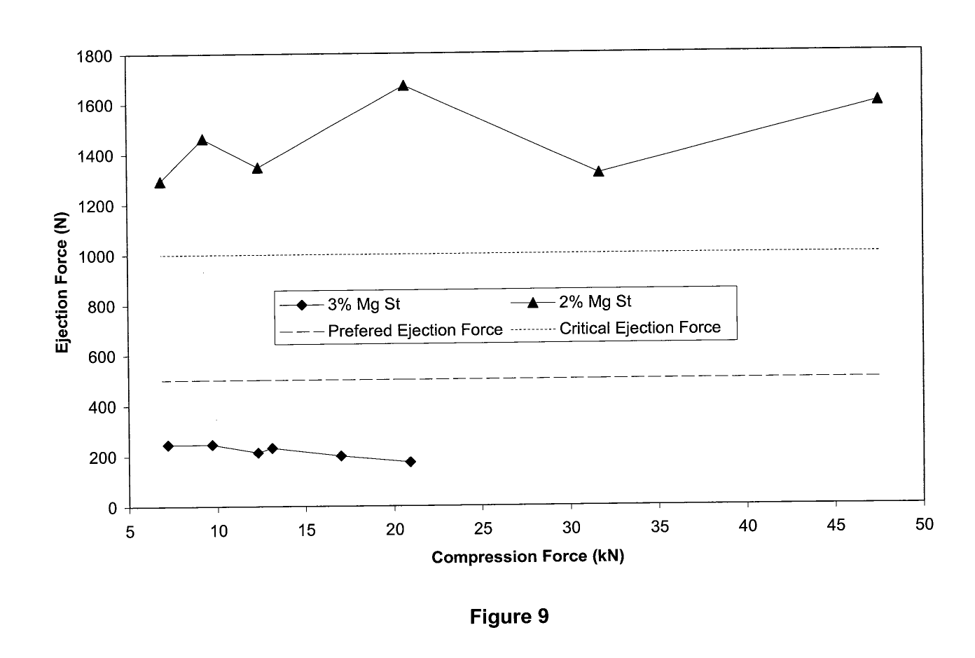

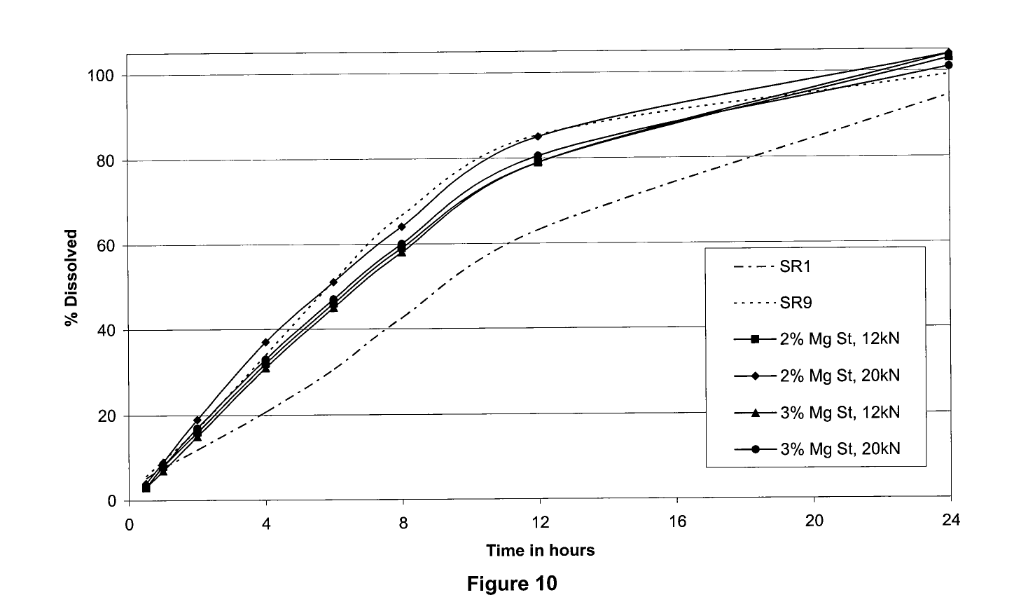

Tablets (600 mg) were prepared using Blend D and Blend E and the ejection force determined. As shown in FIG. 9, increasing the amount of magnesium stearate from 2 wt-% (Blend D) to 3 wt-% (Blend E) reduced the force needed to eject a tablet from a compression tool. Dissolution profiles for the two blends are shown in FIG. 10.

### Example 12

### Chemical Stability of Compound (1) in Tablet Formulations

Open dish chemical stability of compound (1) under various conditions of temperature and humidity were determined for different tablet formulations. The tablets were exposed to temperature and humidity for up to 3 months, and the amount of compound (1) and lactam metabolites, 3-azaspiro[4.5]decan-2-one, lactam (1); 3-[3-methyl-1-methylene-5-(methylethyl)hex-5-enyl]-3-azaspiro[4.5]decan-2-one, lactam (2); and 3-[(3-oxo-2-azaspiro[4.5]dec-2-yl)ethyl]-3-azaspiro[4.5]decan-2-one, lactam (3) were determined. The composition of the tablet formulations containing granules prepared by high shear wet granulation was: 95.5 wt-% granules comprising 98 wt-% compound (1), 1 wt-% METHOCEL™-E4M, and 1 wt-% SLS; and blended with 3 wt-% METHOCELT™-K100M, and 1.5 wt-% magnesium stearate. The SR1, SR4, and SR9 formulations were prepared according to Example 5 using compound (1) from different synthetic lots. The results are presented in Table 11 and Table 12. The high drug loading tablet formulations showed lower amounts of lactam degradants compared to tablet formulations prepared using roller compaction (SR1, SR4, and SR9).

TABLE 11
Open dish stability of 1200 mg dose tablets at
40° C./43% RH for 17 days.

| | High Loading | | | |
|---|---|---|---|---|
| Compound | Tablet | SR1 | SR4 | SR9 |
| Compound (1) (wt-%) | 96.7 | 96.6 | 98.2 | 98.5 |
| Lactam (1) (wt-%) | 0.09 | 0.20 | 0.16 | 0.11 |
| Lactam (2) (wt-%) | 0.02 | 0.05 | 0.04 | 0.03 |
| Lactam (3) (wt-%) | 0.03 | 0.11 | 0.09 | 0.04 |
| Total lactam (wt-%) | 0.14 | 0.36 | 0.29 | 0.18 |

TABLE 12
Open dish stability of 1200 mg dose tablets at
40° C./75% RH for 17 days.

| | High Loading | |
|---|---|---|
| Compound | Tablet | SR4 |
| Compound (1) (wt-%) | 94.4 | 91.1 |
| Lactam (1) (wt-%) | 0.56 | 2.41 |
| Lactam (2) (wt-%) | 0.11 | 0.59 |
| Lactam (3) (wt-%) | 0.27 | 1.42 |
| Total lactam | 0.94 | 3.42 |

### Example 13

### Pharmacokinetics of Gabapentin in Human Subjects Following Administration of Sustained Release Oral Dosage Forms

The pharmacokinetics of gabapentin following administration of sustained release dosage forms provided by the present disclosure was determined using an open label, five-period study of 10 healthy adult male volunteers under fasted conditions, with a 5-day wash out between treatments. In Period 1 of the study, all subjects received a single oral dose of 600 mg (2×300 mg) of NEURONTIN® (gabapentin). In the subsequent four periods (Periods 2 through 5) each subject received one of two different compound (1) SR tablet formulations in a random order. All compound (1) formulations were administered at a single oral dose of 1200 mg compound (1) (two 600 mg tablets). The two compound (1) tablet formulations were the SR1 and SR9 formulations prepared according to Example 5.

Blood samples (approximately 3 mL) were collected from all subjects prior to dosing, and at certain times after dosing into tubes containing K₂EDTA. Within 15 minutes of collection, blood samples were centrifuged for 15 minutes at 3000 rpm at approximately 4° C. Two aliquots of plasma (˜0.7 mL each) were transferred by pipette to VWR tubes. Samples were stored at −20° C. or lower prior to analysis.

Complete urine collections were obtained prior to dosing, and at the 0-4 hr, 4-8 hr, 8-12 hr, 12-24 hr, and 24-36 hr intervals after dosing. The total urine volume was measured at each interval by weight (g) and converted to mL using a 1 g=1 mL relationship. Two aliquots of urine sample (1.5 mL each) were stored at −20° C. or lower prior to analysis.

Plasma samples were analyzed by a validated LC-MS/MS method for the determination of gabapentin in human plasma (K₂EDTA). The method was linear over the concentration range from 80 to 10,000 ng/mL The intra-batch precision (% CV) was 2.83% and the intra-batch accuracy (% theoretical) ranged from 97.5 to 104%; the inter-batch precision was ≦5.76% and the inter-batch accuracy ranged from 97.7 to 104%.

Urine samples were analyzed by a validated LC-MS/MS method for the determination of gabapentin in human urine. The method was linear for gabapentin over the concentration range from 50 to 12,500 ng/mL. The intra-batch precision (% CV) was ≦4.93% and the intra-batch accuracy (% theoretical) ranged from 96.0 to 102%; the inter-batch precision was ≦5.14% and the inter-batch accuracy ranged from 98.1 to 105%. Concentration data for gabapentin in plasma were analyzed by noncompartmental methods using WINNONLIN™ (WinNonlin™ Software version 4.1., Pharsight Corporation, Mountain View, Calif.).

All concentration values below the limit of quantitation were treated as 0 (zero) for the pharmacokinetic analysis and the statistical calculation. Actual time points were used for the calculation of pharmacokinetic parameters. All concentration data and PK parameters were plotted using SIGMAPLOT™ (Version 9.0, Systat Software Inc, Point Richmond, Calif.).

The maximum concentration (Cₘₐₓ) and time to Cₘₐₓ (Tₘₐₓ) were obtained by observation. The apparent elimination half-life (T₁₁₂) was obtained by linear regression of three or more log-transformed data points in the terminal phase. The area under the concentration versus time curve (AUC) was obtained by the linear trapezoidal method using concentration data over the dosing interval. The AUC value extrapolated to infinity (AUC_{inf}) was calculated as:

AUC_{inf}=AUC₍₀₋ₜₗₐₛₜ₎±Cₗₐₛₜ/λ_{z}

where tₗₐₛₜ is the time of the last quantifiable concentration (Cₗₐₛₜ) and λ_{z} is the rate bast constant of the apparent terminal elimination phase.

The total amount of gabapentin excreted during each urine collection period (Ae) was calculated as:

Ae₍ₜ₁₋ₜ₂₎=C₍ₜ₁₋ₜ₂₎×V₍ₜ₁₋ₜ₂₎

where Ae₍ₜ₁₋ₜ₂₎ is the amount excreted in mg over the time interval t1 to t2, C₍ₜ₁₋ₜ₂₎ is the concentration in mg/mL of analyte in the urine collected over this interval, and V₍ₜ₁₋ₜ₂₎ is the total volume of the urine sample in mL. The total amount excreted over 36 hours (Ae₍₀₋₃₆₎) was calculated as the sum of the amounts excreted in all intervals. The percent of the dose excreted in 36 hours as gabapentin was calculated as:

Percent Dose Excreted (% R)=100*(Ae₍₀₋₃₆₎/D)

where D is the administered dose of compound (1) expressed in mg-equivalents of gabapentin. Since gabapentin is excreted exclusively in urine without significant metabolism, the urinary recovery of gabapentin after oral dosing is equivalent to the oral bioavailability (F). This assumption requires accurate measurement of urine volumes for each collection period.

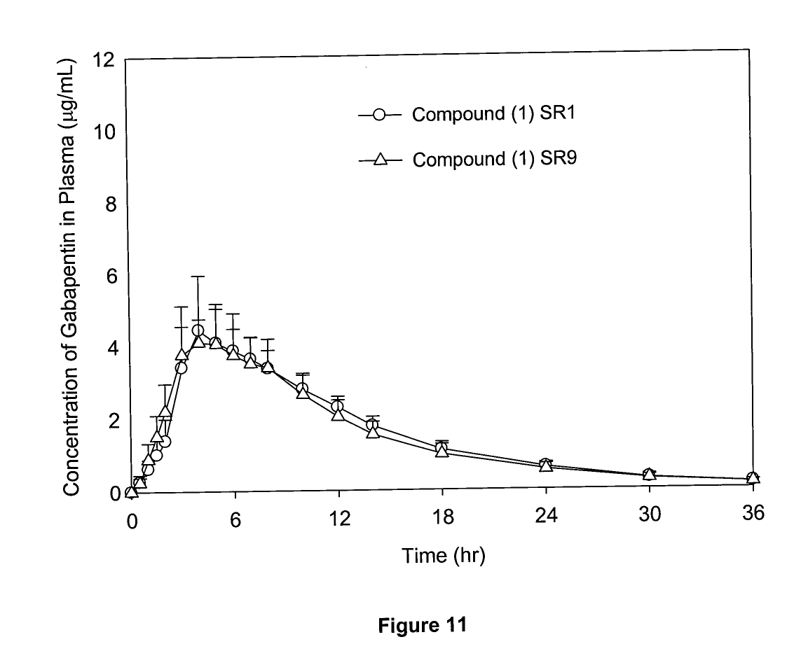

Pharmacokinetic profiles of gabapentin in the plasma of healthy patients following administration of a single oral dose of 1200 mg compound (1) (two 600 mg tablets) as SR1 or SR9 tablets is shown in FIG. 11 and the parameters are summarized in Table 13.

TABLE 13
Mean pharmacokinetic parameters for gabapentin in plasma after oral dosing
of 1200 mg compound (1) SR tablet formulations to ten (10) fasted healthy adult male
subjects.

| | | Dose | | | | | | | |
|---|---|---|---|---|---|---|---|---|---|
| | Dose | (mg-eq. | Cₘₐₓ | Tₘₐₓ | T_{1/2} | C₁₂ | | AUC_{inf} | F |
| Treatment | (mg) | gabapentin) | (μg/mL) | (hr) | (hr) | (μg/mL) | Cₘₐₓ/C₁₂ | (μg × hr/mL) | (%) |
| SR1 | 1200 | 625 | 4.82 | 4.63 | 6.05 | 2.28 | 2.11 | 54.7 | 56.1 |
| SR9 | 1200 | 625 | 4.51 | 4.90 | 6.29 | 2.00 | 2.26 | 52.9 | 52.0 |

Abbreviations:
GP = gabapentin;
Cₘₐₓ = maximum concentration;
Tₘₐₓ = time to Cₘₐₓ;
T_{1/2} = half-life;
AUC = area under the concentration-time curve:
F = bioavailability based on urinary recovery.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

What is claimed is:

1. A tablet dosage form comprising about 80 wt-% to about 95 wt-% 1-([α-isobutanoyloxyethoxy)carbonyl]aminomethyl)-1-cyclohexane acetic acid or a pharmaceutically acceptable salt thereof.

2. The tablet dosage form of claim 1, comprising about 300 mg to about 1300 mg 1-([(α-isobutanoyloxyethoxy)carbonyl]aminomethyl)-1-cyclohexane acetic acid.

3. The tablet dosage form of claim 1, wherein the 1-([α-isobutanoyloxyethoxy)carbonyl]aminomethyl)-1-cyclohexane acetic acid is in free acid form.

4. The tablet dosage form of claim 3, wherein the 1-([(α-isobutanoyloxyethoxy)carbonyl]aminomethyl)-1-cyclohexane acetic acid in free acid form is crystalline.

5. The tablet dosage form of claim 1, comprising hydroxypropylmethyl cellulose and a lubricant.

6. The tablet dosage form of claim 5, comprising:
about 3 wt-% to about 15 wt-% of the hydroxypropylmethyl cellulose; and
about 2 wt-% to about 3 wt-% of the lubricant.

7. The tablet dosage form of claim 6, wherein the hydroxypropylmethyl cellulose is a hypromellose 2208 polymer having a methoxyl content of 19% to 24%, a hydroxypropyl content of 7% to 12%, and a viscosity of 80,000 cps to 120,000 cps in a 2% aqueous solution.

8. The tablet dosage form of claim 1, comprising granules.

9. The tablet dosage form of claim 8, wherein the granules comprise greater than about 95 wt-% 1-([α-isobutanoyloxyethoxy)carbonyl]aminomethyl)-1-cyclohexane acetic acid or a pharmaceutically acceptable salt thereof.

10. The tablet dosage form of claim 9, wherein the 1-([α-isobutanoyloxyethoxy)carbonyl]aminomethyl)-1-cyclohexane acetic acid is in free acid form and is crystalline.

11. The tablet dosage form of claim 10, wherein the granules comprise:
a surfactant; and
a hydroxypropylmethyl cellulose polymer.

12. The tablet dosage form of claim 11, comprising:
about 0.5 wt-% to about 2 wt-% of the surfactant; and
about 0.5 wt-% to about 2 wt-% of the hydroxypropylmethyl cellulose polymer.

13. The tablet dosage form of claim 12, wherein the hydroxypropyl methyl cellulose polymer is hypromellose 2910 polymer having a methoxyl content of 28-30%, a hydroxypropyl content of 7-12%, and a viscosity of 3,000 cps to 5,600 cps in a 2% aqueous solution.

14. The tablet dosage form of claim 12, wherein the granules consist essentially of:
about 98 wt-% 1-([(α-isobutanoyloxyethoxy)carbonyl]aminomethyl)-1-cyclohexane acetic acid;
about 1 wt-% of the surfactant; and
about 1 wt-% of the hydroxypropylmethyl cellulose polymer, which is a hypromellose 2910 polymer having a methoxyl content of 28-30%, a hydroxypropyl content of 7-12%, and a viscosity of 3,000 cps to 5,600 cps in a 2% aqueous solution.

15. The tablet dosage form of claim 1, wherein the dosage form is a sustained release dosage formulation.

16. The tablet dosage form of claim 1, wherein release of the 1-([α-isobutanoyloxyethoxy)carbonyl]aminomethyl)-1-cyclohexane acetic acid from the oral dosage form exhibits the following dissolution profile in 10 mM, pH 7.4, potassium phosphate monobasic buffer with 1% sodium lauryl sulfate at 37° C. stirred at 50 rpm (USP, Type II):
about 26% to about 41% of the 1-([α-isobutanoyloxyethoxy)carbonyl]aminomethyl)-1-cyclohexane acetic acid is released at about 4 hours;
about 50% to about 78% of the 1-([α-isobutanoyloxyethoxy)carbonyl]aminomethyl)-1-cyclohexane acetic acid is released at about 8 hours;
about 68% to about 100% of the 1-([α-isobutanoyloxyethoxy)carbonyl]aminomethyl)-1-cyclohexane acetic acid is released at about 12 hours; and
about 95% to about 100% of the 1-([(α-isobutanoyloxyethoxy)carbonyl]aminomethyl)-1-cyclohexane acetic acid is released at about 20 hours.

17. The tablet dosage form of claim 1, wherein release of the 1-([α-isobutanoyloxyethoxy)carbonyl]aminomethyl)-1-cyclohexane acetic acid from the oral dosage form exhibits the following dissolution profile in 10 mM, pH 7.4, potassium phosphate monobasic buffer with 1% sodium lauryl sulfate at 37° C. stirred at 50 rpm (USP, Type II):
about 30% to about 36% of the 1-([α-isobutanoyloxyethoxy)carbonyl]aminomethyl)-1-cyclohexane acetic acid is released at about 4 hours;
about 56% to about 68% of the 1-([(α-isobutanoyloxyethoxy)carbonyl]aminomethyl)-1-cyclohexane acetic acid is released at about 8 hours;
about 76% to about 94% of the 1-([α-isobutanoyloxyethoxy)carbonyl]aminomethyl)-1-cyclohexane acetic acid is released at about 12 hours; and
about 85% to about 100% of the 1-([α-isobutanoyloxyethoxy)carbonyl]aminomethyl)-1-cyclohexane acetic acid is released at about 20 hours.

18. The tablet dosage form of claim 1, exhibiting a dissolution profile in 10 mM, pH 7.4, potassium phosphate monobasic buffer with 1% sodium lauryl sulfate at 37° C. stirred at 50 rpm (USP, Type II), which when compared with a dissolution profile in which about 33% of the 1-([(α-isobutanoyloxyethoxy)carbonyl]aminomethyl)-1-cyclohexane acetic acid is released at about 4 hours; about 62% of the 1-([α-isobutanoyloxyethoxy)carbonyl]aminomethyl)-1-cyclohexane acetic acid is released at about 8 hours; about 85% of the 1-([(α-isobutanoyloxyethoxy)carbonyl]aminomethyl)-1-cyclohexane acetic acid is released at about 12 hours; and about 92% of the 1-([α-isobutanoyloxyethoxy)carbonyl]aminomethyl)-1-cyclohexane acetic acid is released at about 20 hours, provides an f₁ difference factor less than 15 and an f₂ similarity factor from 50 to 100.

19. The tablet dosage form of claim 1, comprising about 1200 mg compound (1) and when administered to a population of 10 healthy, fasted adult male patients provides a mean gabapentin plasma concentration profile characterized by a Cₘₐₓ from about 3.73 μg/mL to about 5.83 μg/mL and an AUC_{mf} from about 43.1 μg×hr/mL to about 67.3 μg×hr/mL.

20. The tablet dosage form of claim 1, comprising less than about 0.2 wt-% lactam, where lactam wt-% is relative to the nominal amount of 1-([α-isobutanoyloxyethoxy)carbonyl]aminomethyl)-1-cyclohexane acetic acid in the dosage form.

21. The tablet dosage form of claim 20, wherein the dosage form comprises less than about 0.2 wt-% lactam following exposure to 40° C. and 43% relative humidity for at least about 17 days.

22. The oral dosage form of claim 1, having a friability less than about 0.5 wt-% determined according to USP 1216.

23. A solid granulation comprising greater than about 95 wt-% 1-([α-isobutanoyloxyethoxy)carbonyl]aminomethyl)-1-cyclohexane acetic acid or pharmaceutically acceptable salt thereof.

24. The solid granulation of claim 23, wherein the granulation exhibits a Flodex less than about 20 mm.

25. The solid granulation of claim 23, wherein the granulation is prepared by high speed wet granulation.

26. An oral dosage form comprising the granulation of claim 23.

27. The oral dosage form of claim 26, wherein the granulation is compressed into a tablet.

28. A method of treating a disease in a patient wherein the disease is chosen from epilepsy, essential tremor, chronic regional pain syndrome, fibromyalgia, radiculopathy, abdominal-visceral pain, irritable bowel syndrome, migraine, generalized anxiety disorder, depression, insomnia, overactive bladder, hot flashes, premature ejaculation, restless legs syndrome, neuropathic pain, chronic lower back pain, alcohol dependency, complex regional pain syndrome, post-operative pain, cancer-induced pain, bipolar disorder, social anxiety disorder, Parkinson's disease, asthma, cough, chronic obstructive pulmonary disease, and vulvodynia, comprising orally administering to a patient in need of such treatment at least one tablet dosage form of claim 1.

29. The method of claim 28, wherein the disease is restless legs syndrome.

30. The method of claim 28, wherein the disease is neuropathic pain.
